(19) United States
(12) Patent Application Publication
(10) Pub. No.: US 2023/0151377 A1
(43) Pub. Date: May 18, 2023

(54) PLANTS WITH IMPROVED GROWTH

(71) Applicant: SWETREE TECHNOLOGIES AB, Umeå (SE)

(72) Inventors: David JONSÉN, Umeå (SE); Magnus HERTZBERG, Umeå (SE); Anna KARLBERG, Umeå (SE); Thomas MORITZ, Umeå (SE); Maria ERIKSSON, Umeå (SE)

(73) Assignee: SWETREE TECHNOLOGIES AB, Umeå (SE)

(21) Appl. No.: 16/346,487

(22) PCT Filed: Oct. 30, 2017
(86) PCT No.: PCT/SE2017/051065

§ 371 Date: Apr. 30, 2019

(30) Foreign Application Priority Data
Oct. 31, 2016 (SE) 1651431-7

(51) Int. Cl. C12N 15/82

(52) U.S. Cl. CPC C12N 15/8229; C12N 15/8297; C12N 15/8262

(57) ABSTRACT

The present invention relates to genetically modified woody plants comprising a heterologous nucleic acid construct comprising a promoter sequence operably linked to a coding sequence encoding a gibberellin 20-oxidase gene product, wherein the promoter is preferentially or specifically expressed in meristematic tissue of said plant. The invention further relates to methods for producing such plants and to certain nucleic acid molecules useful as promoters.

### CROSS REFERENCE TO RELATED APPLICATIONS

This application is a U.S. National Phase patent application under 35 U.S.C. § 371 of PCT/SE20I7/051065, filed Oct. 30, 2017, which claims priority to Sweden Patent Application No. SE 1651431-7, filed Oct. 31, 2016, the disclosures of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 616562024700SEQLIST.TXT, date recorded: Apr. 19, 2019, size: 82 KB).

### FIELD OF THE INVENTION

The invention relates to the field of plants with improved growth properties, and in particular to plants comprising heterologous nucleic acid constructs comprising improved combinations of gibberellin 20-oxidase genes and promoters influencing their expression in the plants.

### BACKGROUND TO THE INVENTION

Plant growth is regulated by different growth hormones of which one is gibberellic acid, GA. Gibberellins (GAs) are a group of more than 100 tetracyclic diterpenes, some of which are essential endogenous regulators that influence growth and development processes throughout the plant life cycle, e.g. shoot elongation, the expansion and shape of leaves, flowering and seed germination. Several examples illustrating the importance of GAs for regulating growth can be found in the literature. At the cellular level GAs have been found to promote both cell division and cell elongation. Expression of GA in plants is mainly found in growing parts of the plants.

### Plant Growth

Growth of plants appear at apical meristems and results in the development of sets of primary tissues and in the lengthening of the stem and roots. In addition to this primary growth, trees undergo secondary growth and produce secondary tissue “wood” from the cambium. This secondary growth increases the girth of stems and roots. There are several factors such as different gene products that might need to be altered in order to enhance biomass production in trees. Growth in height, diameter, stem volume and wood density are important traits to observe for increased growth and biomass production.

It is known to a person skilled in the art that the phenotypical effect of any gene in the plant is highly dependent on gene regulation. For example, spatial and temporal expression patterns as well as stress induction of genes significantly influence the plant phenotype. Conversely, controlling gene regulation can be used in attempts to improve the plant phenotype, for example, increasing plant growth. Gene expression can be modified using promoters which spatially and temporally direct gene expression in specific tissues and to specific levels. Positive phenotypical traits conferred by a gene can be modified to improve growth by controlling gene expression. Similarly, controlling gene regulation can also be used to attempt to prevent negative phenotypical effects of a gene.

However, it is also known to a person skilled in the art that a specific spatial and temporal expression pattern of a gene may elicit different phenotypical effects under two distinctly different growth conditions, for example, the growth conditions to which the plants are exposed in the greenhouse compared to in a field trial environment.

### Promoters

Promoters are regions of DNA involved in binding of RNA polymerase to initiate transcription of coding sequences. Promoters can comprise several regulatory elements, usually called cis elements, generally located within a few hundred nucleotides from the transcription initiation site but that may also be positioned as far upstream as several thousand nucleotides as well as in introns. Trans-acting proteins then usually bind these cis elements and then regulate transcription. The cis regulatory elements are separated along the nucleotide sequence by nucleic acid stretches that have no regulatory effect on their own, the spacing of the cis-elements could however be important for their function.

Promoters may be constitutive, rhythmic, tissue-specific, or inducible by certain stimuli.

Constitutive promoters induce expression of the coding sequence in most tissues of the plant, irrespective of developmental stage or environmental factors.

Tissue-specific promoters induce expression of the coding sequence in a specific tissue or region of the plant.

Rhythmic promoters is subjected to internal rhythms by an internal timer, these internal timers are for example influenced by light and temperature and their status influence long term expression patterns, for example yearly variations in gene expression.

Promoters can also have temporal variations in activity, for example could the activity of a promoter be reduced or increased during flower induction or dormancy related processes.

Inducible promoters are activated by chemical or physical factors, such as Isopropyl β-D-1-thiogalactopyranoside (IPTG), light, or temperature.

The CaMV 35S promoter is the most frequently used promoter when studying effects of modified gene expression during development, since the studied genes are constitutively expressed when the promoter is operably linked to them. The use of the 35S promoter has generated a lot of data regarding gene function and effects of over-expression in laboratory tests. In some situation it can be useful to have access to a promoter that in combination with a gene is more specifically expressed in a certain plant tissue or plant part. Results from field tests have shown that trees genetically modified with a construct with the 35S promoter operably linked to a trait gene may be acceptable, but have also been shown to result in unimproved or adverse effects in the field.

Results from field tests have shown that plants genetically modified with a construct with the 35S promoter operably linked to a trait gene may be acceptable, but have also been shown to result in unimproved or adverse effects in the field.

### Wood Production

Wood is used for paper production and for constructions. In many situations there is a need for improved properties and improved quality of the wood used. The main need is the quantity of wood. This can be achieved by cutting down more trees, or by using more land for tree production or by using trees which grow faster and have better growth properties. The later can be done by traditional breeding programs or by use of gene modification. Both strategies lead to a shorter rotation time, i.e. the time from planting to harvest. A major disadvantage with traditional tree breeding, especially for forest tree species, is the slow progress due to their long generation periods. Breeding programs are also dependent on the genetic variation present in a tree population. However, by taking advantage of recent developments in gene technology the time required to produce a new variety could be reduced significantly and the effect could be additive to effects produced by breeding.

### Gibberellins

Gibberellins (GAs) are a group of more than 100 tetracyclic diterpenes, some of which are essential endogenous regulators that influence growth and development processes throughout the plant life cycle, including shoot elongation, the expansion and shape of leaves, flowering, and seed germination.

The molecular biology and biochemistry of GA have been extensively reviewed, Busov et al. 2008, New Phytol 177(3):589-607 and Sponsel and Hedden 2010, in, Plant Hormones, Springer Netherlands, pp 63-94, as two examples.

The best examples illustrating the importance of GAs in control of shoot elongation are GA-deficient mutants of Arabidopsis, maize, and pea. These have reduced levels of active GA(s) compared to wild type plants, resulting in a dwarfed phenotype due to a reduction in internode length Sponsel and Hedden 2010 in: Plant Hormones, Springer Netherlands, pp 63-94.

Gibberellin 20-oxidase (GA20ox) is a multifunctional enzyme, a key enzyme, in controlling GA biosynthesis. It catalyses the stepwise conversion of the C-20 gibberellins to C-19 gibberellins. In EP1261726, it is shown that a DNA sequence coding for the expression of a polypeptide exhibiting GA 20-oxidase activity under the control of the 35S promoter inserted in the tree genome results in increased biomass production, improved growth and have more numerous and longer xylem fibres than unmodified wild type plants. These findings have also been published in Eriksson et al. 2000, Nature Biotechnology, 18: 784-788. This article also show that using the promoter CaMV 35S promoter linked to the Arabidopsis thaliana GA 20-oxidase 1 gene, AtGA20ox1, in hybrid aspen (Populus tremula x P. tremuloides) improves growth rate and biomass in transgenic hybrid aspen. They also show that 35S:AtGA20ox1 has an antagonistic effect on root initiation, as the transgenic lines showed poorer rooting than the control plants when potted in soil. Strong constitutive over-expression of the AtGA20ox1 gene by use of the 35S promoter also results in an increase in wood formation mediated by GA signalling, Mauriat and Moritz, 2009, The plant Journal 58, 989-1003. These results show that the GA 20-oxidase gene can be used to promote both primary and secondary growth in the transgenic plant.

Lu et al., 2015, Tree Genetics & Genomes 11: 127, transformed a model poplar genotype (Populus tremula x P. alba) with seven promoters, whereof four novel promoters came from Populus trichocarpa, and five genes. The four novel promoters were cloned from 1.5 to 3 kb sequence fragments upstream of Populus trichocarpa genes. In addition, two previously known promoters from poplar and the 35S promoter were used. The promoters were operatively linked to five different genes, to produce eight constructs. The studied GA 20-oxidase genes were PtGA20ox7 and PtGA20ox2-2, and their effects were measured under greenhouse and field conditions. In field tests some growth improvement was noted, one of the tested constructs showed greater growth improvement. The greenhouse and field responses were highly variable. These experiments did not test promoters preferentially expressed in cambium.

Transgenic hybrid poplar trees (P. alba x P. tremula var. glandulosa clone BH) with two different promoters, 35S and the developing xylem tissue-specific promoter DX15, have been linked to gibberellin 20-oxidase 1, PdGA20ox1, from Pinus densiflora by Jeon et al. 2016, Plant Biotechnology Journal 14, pp. 1161-1170. The DX15 promoter is not expressed in the cambium, Ko et al 2012, Plant Biotechnology Journal 10, pp. 587-596. These transgenic poplar trees showed a three time increase in biomass with accelerated stem growth and xylem differentiation. It should be noted that the control plants in this study had unusually short internodes and stunted growth, a phenotype that depending on cause could be rescued by increased GA levels. Undesirable phenotypes of these poplar were poor root growth and leaf development.

When specifically over-expressing the AtGA20ox1 gene in the xylem of a fast-growing hybrid aspen clone (Populus tremula L. x tremuloides Michx., clone T89) by use of the xylem-expressed LMX5 promoter, the height of the transgenic plants does not significantly differ from wild type. Furthermore, pLMX5-AtGA20ox1 plants do not show any increase in wood formation (Mauriat and Moritz, 2009, The Plant Journal 58, 989-1003).

Specifically expressing the AtGA20ox1 gene in the phloem of T89 hybrid aspen trees, by use of the pLMP1 promoter, described in WO2004097024, does not significantly increase height growth or biomass production of the transgenic plant compared to wild type (Moritz, unpublished results).

WO2011/065928 disclose the use of the promoter of a Cinnamyol CoA reductase (CCR) gene as a vascular specific promoter in combination with a gibberellin 20 oxidase gene from Arabidopsis for producing transgenic plants, e.g. tobacco plants. The effect of the promoter-gene combination in woody plants was not investigated. This promoter is not known to be preferentially expressed in the cambium.

As previously described, multiple sources report on pleiotropic effects of strong constitutive over-expression of the GA 20-oxidase gene that negatively affects the transgenic plant. Eriksson et al. 2000, Nature Biotechnology, 18: 784-788 as well as Jeon et al. 2016, Plant Biotechnology Journal 14, pp. 1161-1170 describes negative effects on root initiation and growth of the transgenic plants over-expressing GA 20-oxidase by use of the 35S promoter. These plants also show strongly impaired seasonal growth responses, Eriksson et al. 2015, New Phytologist, 205: 1288-1295.

In conclusion, to anticipate the effect that a specific promoter-gene combination has on the plant is ingenious and nontrivial. Prior art does not provide information enough to foresee the effect that a specific combination of promoter and GA 20-oxidase gene will have on the plant. Nor does prior art provide information enough to indicate which promoter should be used in combination with the GA 20-oxidase gene to improve plant growth and biomass production without the negative pleiotropic effects that strong constitutive 35S promoter expression may induce.

### BRIEF DESCRIPTION OF THE DRAWINGS

FIG. 1, shows greenhouse and field trial data for a prior art hybrid aspen, wherein a trait gene is expressed under the constitutive 35S promoter.

### SUMMARY OF THE INVENTION

In some situations, it can be useful to have access to a promoter that in combination with a gene, is specifically expressed in a specific plant tissue or plant part. Thus there is a need for new combinations of new functional promoters in combinations with genes that are well functional in field use, i.e. when the plant is grown under realistic outdoor conditions, such as in the real environment of the plant of interest. The present invention builds on the idea that a weak but specific promoter showing desired results on the wanted phenotype, when operably linked to a GA 20-oxidase gene, will give less pleiotropic and possibly less negative effects in the field and in the mass production of a selected transgenic tree.

Thus there is a need for new combinations of functional promoters in combinations with genes that are well functional in field use, i.e. when the plant is grown under realistic outdoor conditions, such as in the real environment when growing the plant of interest.

In view of the need to provide plants capable of enhanced growth and biomass in a range of different environmental conditions, as well as changing environmental conditions, there is a continual need to provide plants with different genetic traits, comprising different sets of promoters and active genes.

Furthermore, in view of the need to provide trees capable of enhanced growth and biomass in a range of different environmental conditions, as well as changing environmental conditions, there is a continual need to provide trees with different genetic traits, comprising different sets of promoters and active genes.

Thus, in a first aspect the invention relates to genetically modified plants comprising a heterologous nucleic acid construct comprising a promoter sequence operably linked to a coding sequence encoding a gibberellin 20-oxidase gene product, wherein the promoter is preferentially or specifically expressed in meristematic tissue of said plant.

In another aspect the invention relates to genetically modified woody plants comprising a heterologous nucleic acid construct comprising a promoter sequence operably linked to a coding sequence encoding a gibberellin 20-oxidase gene product, wherein the promoter is preferentially or specifically expressed in meristematic tissue of said plant.

In one embodiment, the promoter is preferentially or specifically expressed in at least one of cambium, vascular meristematic tissue, and shoot meristem tissue of said plant.

In one embodiment, the promoter is not significantly expressed in at least one of mature xylem, stem phloem, whole leaves, whole roots and bark of said plant.

In one embodiment, the promoter is selected from the group consisting of pEC1 (SEQ ID NO: 7, 26, or 31), pAIL1 (SEQ ID NO: 10 or 29), pEA2 (SEQ ID NO: 4 or 23), and promoters that have the same, or essentially the same, capability of initiating transcription of a coding sequence when operably linked to said coding sequence.

In one aspect, the invention relates to genetically modified woody plants comprising a heterologous nucleic acid construct comprising a promoter sequence operably linked to a coding sequence encoding a gibberellin 20-oxidase gene product, wherein the promoter is selected from the group consisting of pEC1 (SEQ ID NO: 7, 26, or 31), pAIL1 (SEQ ID NO: 10 or 29), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), and promoters that have the same, or essentially the same, capability of initiating transcription of a coding sequence when operably linked to said coding sequence.

In one embodiment of the above aspects, the gibberellin 20-oxidase gene product is a gibberellin 20-oxidase type 1 gene product.

In one embodiment of the above aspects, the gibberellin 20-oxidase gene product is a gibberellin 20-oxidase from Arabidopsis thaliana, Eucalyptus grandis, or Populus tremula x tremuloides.

In one embodiment of the above aspects, the gibberellin 20-oxidase gene product shows gibberellin 20-oxidase activity and has an amino acid sequence at least 50%, such as 60%, 70%, 75%, 80%, 85%, 90%, 95%, 99% or 100% identical to an amino acid sequence selected from SEQ ID NOs: 14, 16 and 18.

In one embodiment of the above aspects, the plant has a modified trait as compared to a non-modified tree of the same species, wherein the modified trait is selected from plant height, stem diameter, stem volume, wood density, stem dry weight, bark dry weight, average internode length, number of internodes.

In another embodiment the genetically modified plant provided by the invention is characterized by one or more modified phenotypic features selected from the group consisting of, vegetative growth; biomass production; seed production; seed lipid content; wherein the one or more modified phenotypic features are modified as compared with a corresponding wild-type plant of the same species.

In a preferred embodiment, the modified trait is increased as compared to a wild-type plant of the same species, such as increased as compared to a wild-type plant of the same species when said plants are grown under identical field conditions for a period of at least one year.

In one embodiment, the genetically modified plant is a crop plant, for example sugarcane, pumpkin, maize (corn), wheat, rice, barley, rye, rape, oil seed rape, forage grass, beet, cassava, soybean, potato and cotton.

In some embodiments of the invention, the plant is a woody plant, such as a hardwood plant, such as of the genus Eucalyptus or Populus.

In one embodiment of the above aspects, the heterologous nucleic acid construct comprises the promoter pEC1, or a promoter that has the same, or essentially the same, capability of initiating transcription of a coding sequence when operably linked to said coding sequence, and the modified trait is at least one of plant height, stem volume, stem dry weight, bark dry weight, internode length, and wood density.

In one embodiment of the above aspects, the heterologous nucleic acid construct comprises the promoter pEA2, or a promoter that has the same, or essentially the same, capability of initiating transcription of a coding sequence when operably linked to said coding sequence, and the modified trait is at least one of stem diameter, stem volume, stem dry weight, and wood density.

In one embodiment of the above aspects, the heterologous nucleic acid construct comprises the promoter pAIL1, or a promoter that has the same, or essentially the same, capability of initiating transcription of a coding sequence when operably linked to said coding sequence, and the modified trait is at least one of plant height, stem diameter, and number of internodes.

In one embodiment of the above aspects, the heterologous nucleic acid construct comprises the promoter pEL1.1, or a promoter that has the same, or essentially the same, capability of initiating transcription of a coding sequence when operably linked to said coding sequence, and the modified trait is at least plant height.

In one embodiment of the above aspects, wherein the heterologous nucleic acid construct comprises the promoter pEL1.2, or a promoter that has the same, or essentially the same, capability of initiating transcription of a coding sequence when operably linked to said coding sequence, and the modified trait is at least one of plant height, stem diameter, and stem volume.

In one embodiment of the above aspects, wherein the heterologous nucleic acid construct comprises the promoter pEA3, or a promoter that has the same, or essentially the same, capability of initiating transcription of a coding sequence when operably linked to said coding sequence, and the modified trait is at least wood density.

The present invention further relates to a method to make a genetically modified plant according to the invention, said method comprising the following steps;
a) providing suitable part of a plant;
b) providing a heterologous nucleic acid construct comprising a promoter sequence operably linked to a coding sequence encoding a gibberellin 20-oxidase gene product, wherein said promoter is preferentially or specifically expressed in meristematic tissue of said plant;
c) introducing the heterologous nucleic acid construct into said suitable part of the plant; and
d) regenerating a genetically modified plant from said suitable part of the plant.

The present invention further relates to a method to make a genetically modified woody plant according to the invention, said method comprising the following steps;
a) providing suitable part of a woody plant;
b) providing a heterologous nucleic acid construct comprising a promoter sequence operably linked to a coding sequence encoding a gibberellin 20-oxidase gene product, wherein said promoter is preferentially or specifically expressed in meristematic tissue of said woody plant;
c) introducing the heterologous nucleic acid construct into said suitable part of the woody plant; and
d) regenerating a genetically modified tree from said suitable part of the woody plant.

The present invention further relates to a method to make a genetically modified woody plant according to the invention, said method comprising the following steps;
a) providing suitable part of a woody plant;
b) providing a heterologous nucleic acid construct comprising a promoter sequence operably linked to a coding sequence encoding a gibberellin 20-oxidase gene product, wherein said promoter is selected from the group consisting of pEC1 (SEQ ID NO: 7, 26, or 31), pAIL1 (SEQ ID NO: 10 or 29), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), and promoters that have the same, or essentially the same, capability of initiating transcription of a coding sequence when operably linked to said coding sequence;
c) introducing the heterologous nucleic acid construct into said suitable part of the woody plant; and
d) regenerating a genetically modified tree from said suitable part of the woody plant.

In a further aspect, the present invention relates to a nucleic acid molecule having the capability to act as a promoter when operably linked to a coding sequence and introduced into a plant, wherein the nucleic acid molecule is selected from the group consisting of:
a) nucleic acid molecules comprising the regulatory elements comprised in the promoter regions pEC1 (SEQ ID NO: 7, 26, or 31), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), pAIL1 (SEQ ID NO: 11 or 29);
b) nucleic acid molecules comprising the promoter region that is located between start codon and 300, 250, 200, 175, 150, or 125 nucleotides upstream of promoter regions pEC1 (SEQ ID NO: 7, 26, or 31), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), pAIL1 (SEQ ID NO: 11 or 29), or nucleic acid stretches that are at least 40%, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to said part of the promoter regions;
c) nucleic acid molecules that are promoters that are orthologous to the promoter regions pEC1 (SEQ ID NO: 7, 26, or 31), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), pAIL1 (SEQ ID NO: 11 or 29);
   wherein said nucleic acid molecule has the same, or essentially the same, capability of initiating transcription of a coding sequence when operably linked to said coding sequence, as compared to the promoter regions pEC1 (SEQ ID NO: 7, 26, or 31), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), pAIL1 (SEQ ID NO: 11 or 29).

In a further aspect, the present invention relates to nucleic acid molecule having the capability to act as a promoter with preferential expression in meristematic tissue when operably linked to a coding sequence and introduced into a woody plant, wherein the nucleic acid molecule is selected from the group consisting of:
a) nucleic acid molecules comprising the regulatory elements comprised in the promoter regions pEC1 (SEQ ID NO: 7, 26, or 31), pEA2 (SEQ ID NO: 4 or 23), pAIL1 (SEQ ID NO: 11 or 29);
b) nucleic acid molecules comprising the promoter region that is located between start codon and 300, 250, 200, 175, 150, or 125 nucleotides upstream of promoter regions pEC1 (SEQ ID NO: 7, 26, or 31), pEA2 (SEQ ID NO: 4 or 23), pAIL1 (SEQ ID NO: 11 or 29), or nucleic acid stretches that are at least 40%, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to said part of the promoter regions;
c) nucleic acid molecules that are promoters that are orthologous to the promoter regions pEC1 (SEQ ID NO: 7, 26, or 31), pEA2 (SEQ ID NO: 4 or 23), pAIL1 (SEQ ID NO: 11 or 29);
   wherein said nucleic acid molecule has the same, or essentially the same, capability of initiating transcription of a coding sequence when operably linked to said coding sequence, as compared to the promoter regions pEC1 (SEQ ID NO: 7, 26, or 31), pEA2 (SEQ ID NO: 4 or 23), pEA3 (SEQ ID NO: 5 or 24), pEL1.1 (SEQ ID NO: 8, 27, or 32), pEL1.2 (SEQ ID NO: 9, 28, or 33), pAIL1 (SEQ ID NO: 11 or 29).

### Definitions

All terms and words used in the present specification are intended to have the meaning generally given to them by the person skilled in the art of plant biotechnology. However, a few terms are explained in more detail below in order to avoid ambiguities.

The nomenclature of genes follow the naming of genes presented at the Phytozome Comparative Plant Genomics Portal (phytozome.jgi.doe.gov) using the latest version of Phytozome. At present the version 11.0 is used. Most gene names in the present disclosure are found in Phytozome. In brief, the first two or three letters denotes the plant name in Latin directly followed by the gene name, exemplified by Gibberellin 20-oxidase type 1 from Arabidopsis thaliana is denoted, AtGA20ox1. The same gene from Eucalyptus grandis is denoted EgGA20ox1.

A “p” in front of a gene denotes that this is the promoter of said gene, for example pRBCS is the promoter of the gene ribulose-1,5-bisphosphate carboxylase small subunit (RBCS).

If a promoter, when operably linked to a coding sequence, entails expression of the coding sequence in a certain tissue or region of the plant to a significantly larger extent than in another tissue or region, then that promoter is said to be “preferentially expressed” in that tissue or region. A promoter may be preferentially expressed in more than one tissue or region. Expression levels can be analysed as described herein.

If a promoter, when operably linked to a coding sequence, entails expression of the coding sequence in a single tissue or region of the plant to a significantly larger extent than in any other tissue or region, then that promoter is said to be “specifically expressed” in that tissue or region. Expression levels can be analysed as described herein.

By “ortholog” or “orthologous polypeptide” is meant a polypeptide expressed by evolutionarily related genes that have a similar nucleic acid sequence, where the polypeptide has similar functional properties. Orthologous genes are structurally related genes, from different species, derived by a speciation event from an ancestral gene. Related to orthologs are paralogs. Paralogous genes are structurally related genes within a single plant species most probably derived by a duplication of a gene. Several different methods are known by those of skill in the art for identifying and defining these functionally homologous sequences.

Orthologous genes from different organisms have highly conserved functions and can be used for identification of genes that could perform the invention in the same way as the genes presented here. Paralogous genes, which have diverged through gene duplication, may encode protein retaining similar functions. Orthologous genes are the product of speciation, the production of new species from a parental species, giving rise to two or more genes with common ancestry and with similar sequence and similar function. These genes, termed orthologous genes, often have an identical function within their host plants and are often interchangeable between species without losing function. Identification of an “ortholog” gene may be done by identifying polypeptides in public databases using the software tool BLAST with one of the polypeptides encoded by a gene. Subsequently additional software programs are used to align and analyze ancestry. The sequence identity between two orthologous genes may be low.

A promoter is said to be an “orthologous promoter” to a promoter in a different species when the respective promoters initiate transcription of orthologous genes in wild type plants of the respective species.

Gibberellin 20-oxidase (GA20ox) is an oxidoreductase involved in the biosynthesis of gibberellins (GAs). It catalyses the stepwise conversion of the C-20 gibberellins GA₁₂/GA₅₃ to C-19 GAs, by three successive oxidations to GA₉ and GA₂₀, which are the immediate precursors of the active gibberellins GA₄ and GA₁, respectively, Coles et al., (1999) The Plant Journal, 17, pp. 547-556. In the ENZYME nomenclature database (http://enzyme.expasy.org/) it is classified in class 1.14.11. Gibberellin 20-oxidase activity can be measured in vitro i.a. according to the assay set out in Gilmour et al., Plant Physiol. 1986 September; 82: 190-195. A protein is considered to show gibberellin 20-oxidase activity if its gibberellin 20-oxidase activity is at least 10%, such as 20%, 30%, 40%, 50%, 60%, 70%, 80%, 90%, 95% or 100% of the gibberellin 20-oxidase activity of a protein having the amino acid sequence according to SEQ ID NO: 16 (PttGA20ox1) in corresponding assays.

A “woody plant” is a plant that produces wood as a structural tissue.

The terms “substantially identical” or “sequence identity” may indicate a quantitative measure of the degree of identity between two amino acid sequences or two nucleic acids (DNA or RNA) of equal length. When the two sequences to be compared are not of equal length, they are aligned to give the best possible fit, by allowing the insertion of gaps or, alternatively, truncation at the ends of the polypeptide sequences or nucleotide sequences. The “sequence identity” may be presented as percent number, such as at least 40, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% amino acid sequence identity of the entire length, when compared and aligned for maximum correspondence, as measured using a sequence comparison algorithm or by visual inspection.

The sequence identity of the polypeptides of the invention can be calculated as (Nref−Ndif)100/Nref, wherein Ndif is the total number of non-identical residues in the two sequences when aligned and wherein Nref is the number of residues in one of the sequences. The sequence identity between one or more sequence may also be based on alignments using the clustalW or ClustalX software. In one embodiment of the invention, alignment is performed with the sequence alignment method ClustalX version 2 with default parameters. The parameter set preferably used are for pairwise alignment: Gap open penalty: 10; Gap Extension Penalty: 0.1, for multiple alignment, Gap open penalty is 10 and Gap Extension Penalty is 0.2. Protein Weight matrix is set on Identity. Both Residue-specific and Hydrophobic Penalties are “ON”, Gap separation distance is 4 and End Gap separation is “OFF”, No Use negative matrix and finally the Delay Divergent Cut-off is set to 30%. The Version 2 of ClustalW and ClustalX is described in: Larkin et al. 2007, Clustal W and Clustal X version 2.0. Bioinformatics, 23:2947-2948.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, and Glutamine.

In some aspects, the amino acid substantial identity exists over a polypeptide sequences length of 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700 amino acids in the polypeptide with a “sequence identity” as defined above.

In certain aspects, substantial identity exists over a region of nucleic acid sequences of at least about 50 nucleic acid residues, such as at least about 100, 150, 200, 250, 300, 330, 360, 375, 400, 425, 450, 460, 480, 500, 600, 700, 800 such as at least about 900 nucleotides or such as at least about 1 kb, 2 kb, or such as at least about 3 kb.

A gene (nucleic acid molecule comprising a coding sequence) is “operably linked” to a promoter when its transcription is under the control of the promoter and where transcription results in a transcript whose subsequent translation yields the product encoded by the gene.

The term “increasing expression” is intended to encompass well known methods to increase the expression by regulatory sequences, such as promoters, or proteins, such as transcription factors. The terms “increasing expression”, “enhanced expression” and “over-expression” can be used interchangeably in this text. Increased expression may lead to an increased amount of the over-expressed protein/enzyme, which may lead to an increased activity of the protein of interest that contributes to its high efficiency.

### DETAILED DESCRIPTION OF THE INVENTION

On a general level, the present invention relates to controlling gene regulation in order to retain or further improve positive phenotypical traits provided by a trait gene when growth conditions change. Controlled gene regulation is used to tailor the expression pattern of the trait gene to the growth condition under which the plant is to be grown.

The present inventors have found that constitutive over-expression of a trait gene that provide improved growth under greenhouse conditions may not provide similar improved growth under field conditions, and may in fact lead to impaired growth (see Example 1).

These unexpected results led the inventors to test other combinations of promoters and genes. It is evident from the results disclosed in Example 1 that having a strong constitutive expression of a trait gene can, as with the 35S promoter construct, have disadvantageous effects under some field trial conditions. Furthermore, these results demonstrate the need for new promoters and new promoter-gene combinations to tailor the expression pattern of the trait gene to the specific growth condition and to retain or further improve the positive phenotypical traits provided by the gene when growth conditions change.

Consequently, the invention consists of combinations of promoters, in particular cambium and leaf promoters, and Gibberellin 20-oxidase type (GA20ox) genes that confer improved tree traits in field use.

### Novel Promoter-Gene Combinations

This invention discloses novel combinations of promoters and trait genes, more specifically the GA20ox gene. When any of these combinations are expressed in a tree a number of improved phenotypical effects are noted, such as plant height, stem diameter, stem volume, wood density, stem dry weight, bark dry weight, average internode length, number of internodes.

The combinations of promoters and genes were designed based on scientific information about the function and expression pattern of the trait gene and the promoter established by the inventors and supported by information available in the prior art. Such information provides concepts where to direct expression as well as where to avert gene expression. However, it is known to a person skilled in the art that anticipating the effect that a specific promoter-gene combination has on the plant is ingenious and nontrivial.

The novel combinations of a promoter and a GA20ox gene is introduced into the plant by use of a recombinant DNA construct, as explained herein.

### Plants

A genetically-modified or transgenic plant cell or plant or a part thereof according to the present invention, that express the novel combinations of a promoter and a GA20ox gene, may be an annual plant or a perennial plant. Preferably the annual or perennial plant is a crop plant having agronomic importance. The annual crop plant can be a monocot plant selected from Avena spp (Avena sativa); Oryza spp., (e.g. Oryza sativa; Oryza bicolour); Hordeum spp., (Hordeum vulgare); Triticum spp., (e.g. Triticum aestivum); Secale spp., (Secale cereale); Brachypodium spp., (e.g. Brachypodium distachyon); Zea spp (e.g. Zea mays); or a dicot plant selected from Cucumis spp., (e.g. Cucumis sativus); Glycine spp., (e.g. Glycine max); Medicago spp., (e.g. Medicago trunculata); Mimulus spp; Brassica spp (e.g. Brassica rapa; Brassica napus; Brassica oleraceae); Camelina spp (e.g. Camelina sativa); Beta vulgaris.

### Woody Plants

The present invention relates to genetically modified woody plants, such as genetically modified angiosperms, dicotyledonous woody plants, preferably trees.

The invention further relates to genetically modified woody plants from gymnosperms, such as conifer trees.

The woody plant may be a hardwood plant e.g. selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants, such as eucalyptus and plants from the Salicaceae family, such as willow, poplar and aspen including variants thereof, are of particular interest, as these groups include fast-growing species of tree or woody shrub which are grown specifically to provide timber for building material, raw material for pulping, bio-fuels and/or bio chemicals.

The woody plant may be a hardwood plant e.g. selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum.

Hardwood plants, such as eucalyptus and plants from the Salicaceae family, such as willow, poplar and aspen including variants thereof, are of particular interest, as these groups include fast-growing species of tree or woody shrub which are grown specifically to provide timber for building material, raw material for pulping, bio-fuels and/or bio chemicals.

In further embodiments, the genetically modified tree is a conifer tree, such as a member of the order Pinales, with members of the family Cupressaceae, such as Cupressus spp., Juniperus spp., Sequoia spp., Sequoiadendron spp.; with members of the family Taxaceae (Taxus spp.) and with members of the family Pinaceae, such as the genera Abies spp., Cedrus spp., Larix spp., Picea spp., Pinus spp., Pseudotsuga spp., Tsuga spp.

Alternatively, the woody plants which may be selected from the group consisting of cotton, bamboo and rubber plants.

In another embodiment, the genetically modified tree is a deciduous trees including hybrids, and cultivars such as acacia (Acacia spp.), alder (Alnus spp.), birch (Betula spp.), hornbeam (Carpinus spp.), hickory (Carya spp.), chestnut (Castanea spp.), beech (Fagus spp.), walnut (Juglans spp.), oak (Quercus spp.), ash (Fraxinus spp.), poplar (Populus spp.), aspen (Populus spp.), willow (Salix spp.), eucalyptus (Eucalyptus spp.), sycamore (Platanus spp.), maple (Acer spp.), mahogany (Swietenia spp.), sweet gum (Liquidambar spp.). Genetically modified trees of the families Salicaceae and Myrtaceae are preferred, most preferred are genetically modified tree from the genus Eucalyptus and Populus.

In yet another embodiment, the genetically modified tree is a fruit bearing plants, including hybrids, and cultivars such as, apple (Malus spp.), plum (Prunus spp.), pear (Pyrus spp.), orange (Citrus spp.), lemon (Citrus spp.), kiwi fruit (Actinidia spp.), cherry (Prunus spp.), grapevine (Vitis spp.), and fig (Ficus spp.).

In a specific embodiment, the genetically modified tree is a woody plant whose leaves can be eaten as leaf vegetables include Adansonia, Aralia, Moringa, Morus, and Toona species.

### Promoters:

This invention has established a number of novel Eucalyptus tissue-specific promoters such as, such as apex active promoters, stem/cambium active promoters and promoters active in leaves. These promoters offer invaluable instruments to specifically control the expression of trait genes in a plant, more specifically in a tree and even more specifically in Eucalyptus.

The novel Eucalyptus promoters were identified by using scientific information available from multiple plant species, such as Eucalyptus, Populus and Arabidopsis, from gene expression analyses, expression of known promoters and the expression and function of the corresponding genes and of identified orthologous/homologous genes.

In order to identify the Eucalyptus promoters a strategy was formulated involving two steps, first identification of a set of promoters and secondly verifying that the identified promoter is functional.

### Identification of Eucalyptus Promoters:

Eucalyptus promoters that corresponds to tested and verified Populus promoters.

Eucalyptus promoters that corresponds to promoters with a well-established expression pattern confirmed by extensive analysis.

Selection of Eucalyptus promoters based on expression pattern analysis performed in Eucalyptus, for example, microarray or RNAseq analysis.

Selection of Eucalyptus promoters based on expression pattern analysis performed in Populus and/or Arabidopsis, for example, microarray or RNAseq analysis.

Once a desired expression pattern was identified a phylogenetic analysis of the corresponding gene and closely related genes from Eucalyptus grandis, Populus trichocarpa and Arabidopsis thaliana was performed using publically available genome database resources. Mostly the Phytozome database was used for searches. Thus, orthology and homology within and between species was determined and a Eucalyptus gene with a putative expression pattern similar to the desired expression pattern was identified.

The region upstream the coding sequence of the identified Eucalyptus gene was examined and a putative promoter region length was determined using available scientific information together with homology analyses of promoter regions of orthologous genes from multiple plant species, such as Eucalyptus, Populus and Arabidopsis.

Eight Eucalyptus promoters were selected for combination with trait genes. A ninth promoter was also included from hybrid aspen, see below for details. The constitutive Cauliflower Mosaic Virus 35S promoter, p35S was combined with all genes for comparison. For details about cloning of the genes, see the examples.

TABLE 1
Eucalyptus and hybrid aspen promoters and the CaMV 35S promoter.

| Promoters | | | Orthologous promoters | | | | | |
|---|---|---|---|---|---|---|---|---|
| | | Promoter | | | Promoter | | | Promoter |
| Promoter | Sequence | region | Promoter | Sequence | region | Promoter | Sequence | region |
| name | ID No. | length (nt) | name | ID No. | length (nt) | name | ID No. | length (nt) |
| pECO1 | 1 | 1084 | pECO1-ort | 20 | 1799 | | | |
| | | | poplar | | | | | |
| pECO2 | 2 | 2000 | pECO2-ort | 21 | 2000 | | | |
| | | | poplar | | | | | |
| pEA1 | 3 | 2000 | pEA1-ort | 22 | 2000 | | | |
| | | | poplar | | | | | |
| pEA2 | 4 | 2500 | pEA2-ort | 23 | 2500 | pAIL1 | 10 | 2683 |
| | | | poplar | | | | | |
| pEA3 | 5 | 2700 | pEA3-ort | 24 | 2700 | | | |
| | | | poplar | | | | | |
| pEA4 | 6 | 2500 | pEA4-ort | 25 | 2500 | pEA4-para | 30 | 2500 |
| | | | poplar | | | poplar | | |
| pEC1 | 7 | 2101 | pEC1-ort | 26 | 2101 | pEC1-para | 31 | 2101 |
| | | | poplar | | | poplar | | |
| pEL1.1 | 8 | 600 | pEL1.1-ort | 27 | 600 | pEL1.1-para | 32 | 600 |
| | | | poplar | | | poplar | | |
| pEL1.2 | 9 | 1800 | pEL1.2-ort | 28 | 1800 | pEL1.2-para | 33 | 1800 |
| | | | poplar | | | poplar | | |
| pAIL1 | 10 | 2683 | pEA2 | 4 | 2500 | pEA2-ort | 23 | 2500 |
| | | | | | | poplar | | |
| p35S | 11 | 942 | | | | | | |

The above identified nucleic acid sequences constitute promoter regions. As known in the art, promoter regions comprise a number of cis-regulatory elements, to which proteins involved in transcription bind. These regulatory elements are primarily located within a few hundred nucleotides upstream the start codon.

Thus, in one aspect the methods and products of the invention make use of the promoter regions in the plants and methods according to the invention.

In further aspects, the methods and products of the invention make use of the regulatory elements comprised in the promoter regions, i.e. polynucleotides that have the same, or essentially the same, capability of initiating transcription of a coding sequence when operably linked to said coding sequence, as compared to the promoter regions disclosed in Table 1.

In one aspect, the methods and products of the invention make use of the part of the promoter region that is located between start codon and 300, 250, 200, 175, 150, or 125 nucleotides upstream, or nucleic acid stretches that are at least 40%, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to said part of the promoter region and that have the same, or essentially the same, capability of initiating transcription of a coding sequence when operably linked to said coding sequence, as compared to the promoter regions disclosed in Table 1.

In one aspect, the methods and products of the invention make use of promoters that are orthologous to the promoters disclosed in Table 1, i.e. promoters from different species that initiate transcription of orthologous genes in wild type plants of the respective species. Also such orthologous promoters should have the same, or essentially the same, capability of initiating transcription of a coding sequence when operably linked to said coding sequence, as compared to the promoter regions disclosed in Table 1.

In one aspect of the invention, the promoter is specifically not a promoter of a cinnamoyl-CoA reductase gene in a wild type plant species.

Assessment of whether a nucleic acid has the same, or essentially the same, capability of initiating transcription of a coding sequence when operably linked to said coding sequence, can be done in a number of ways known to the skilled person. One way is to study expression patterns by histological studies of plants harbouring a promoter-β-glucuronidase (GUS) construct, as detailed in Example 3. The nucleic acid's activity as a promoter is then assayed using the established histochemical GUS staining technique, and compared to one or more constructs harbouring one or more of the promoter regions of the present disclosure.

Promoters from Eucalyptus
The Promoter pECO1

The dynamin protein, a GTPase that is responsible for endocytosis in the eukaryotic cell, was identified as a highly and constitutively expressed gene by studying expression data from hybrid aspen microarray experiments. The promoter to the hybrid aspen gene has been established and used as a constitutive promoter by SweTree Technologies AB. To clone the Eucalyptus pECO1 promoter, the amino acid sequence from Populus trichocarpa dynamin protein gene, accession number Potri.001G090600, was used in a blast search followed by a phylogenetic analysis of the identified putative homologous and orthologous genes. The identified E. grandis ortholog, accession number Eucgr.E00053, has an 86.7% polypeptide sequence identity to the Populus gene product. The sequence immediately upstream of, but not including, the start codon of the gene Eucgr.E00053 was used for synthesis of the pECO1 promoter, Seq ID No: 1. The putative orthologous promoter to the pECO1 promoter is the Populus tremula x tremuloides promoter pECO1-ort poplar, Seq ID No: 20.

The Promoter pECO2

A constitutively expressed gene encoding a housekeeping protein, glyceraldehyde 3-phosphate dehydrogenase, GAPDH, was identified as a constitutively expressed gene suitable as a stable reference for RT-qPCR analysis by Czechowski et al. Plant Physiology 2005, Vol. 139, 5-17. GAPDH catalyses a step in glycolysis and serves to break down glucose for energy and carbon molecules. The GAPDH gene from A. thaliana, accession number AT1G13440, was used in a blast search followed by a phylogenetic analysis of the identified putative homologous and orthologous genes.

The identified Eucalyptus grandis ortholog, accession number Eucgr.H04673, has a 93.1% polypeptide sequence identity to AT1G13440. Avoiding to include the coding region of an adjacent gene, a 1084 base pair long promoter fragment immediately upstream of, but not including, the start codon of gene Eucgr.H04673 was used for synthesis of the pECO2 promoter, Seq ID No: 2. The putative orthologous promoter to the pECO2 promoter is the promoter region, pECO2-ort poplar, Seq ID No: 21, of the Populus trichocarpa gene with accession number Potri.010G055400.

The Promoter pEA1

The gene ERECTA (ER) from A. thaliana (accession number AT2G26330) was selected based on publications regarding its known function and expression in shoot apex. The ER gene is homologous to receptor protein kinases and involved in specification of organs originating from the shoot apical meristem. The ER polypeptide contains a cytoplasmic protein kinase catalytic domain, a transmembrane region, and an extracellular leucine-rich repeat. ER has further been identified as a quantitative trait locus for transpiration efficiency by influencing epidermal and mesophyll development, stomatal density and porosity of leaves. ER has also been implicated in resistance to bacteria and to necrotrophic fungus. ER governs, together with ERL1 and ERL2, the initial decision of protodermal cells to either divide proliferatively to produce pavement cells or divide asymmetrically to generate stomatal complexes. Yokoyama et al. 1998 The Plant Journal, 15(3), 301-310.

The AT2G26330 polypeptide was used in a blast search followed by a phylogenetic analysis of the identified putative homologous and orthologous genes. This identified the E. grandis ortholog, accession number Eucgr.C00732. The orthologous gene of Populus trichocarpa is Potri.006G220100. Since the length of the promoter is unknown, a 2000 base pair long promoter fragment immediately upstream of, but not including, the start codon of gene Eucgr.C00732 was selected for synthesis of the pEA1 promoter, Seq ID No: 3. The putative orthologous promoter to the pEA1 promoter is the promoter region, pEA1-ort poplar, Seq ID No: 22, of the Populus trichocarpa gene with accession number Potri.006G220100.

The Promoter pEA2

The gene AINTEGUMENTA (ANT) from A. thaliana (accession number AT4G37750) was selected for its known function in cell proliferation and as a positive regulator of cell division and for its known expression in actively dividing cells. Loss-of-function Arabidopsis mutants lacking ANT have reduced cell division and cell number leading to reduced size of all lateral organs while overexpression increases cell number and thus organ size, Mizukami and Fischer (2000) PNAS, 97(2): 942-947. The promotor of the Populus ANT homolog, AIL1, is active in actively dividing zones like apex and cambium, Karlberg et al. 2011, PLoS Genetics, 7(11):e1002361.

The AT4G37750 polypeptide was used in a blast search followed by a phylogenetic analysis of the identified putative homologous and orthologous genes. This identified the E. grandis ortholog, accession number Eucgr.F02223. The putative orthologous gene in Populus trichocarpa is Potri.002g114800. Since the length of the promoter is unknown, a 2500 base pair long promoter fragment immediately upstream of, but not including, the start codon of gene Eucgr.F02223 was selected for synthesis of the pEA2 promoter, Seq ID No: 4. The putative orthologous promoter to the pEA2 promoter is the promoter region, pEA2-ort poplar, Seq ID No: 23, of the Populus trichocarpa gene with accession number Potri.002g114800. The pEA2 and pAIL1 are orthologous promoters.

The Promoter pEA3

The promoter of the Asymmetric leaves1 (AS1) gene, accession number AT2G37630, drives gene expression in the apical region of the plant, specifically in the leaf forming tissues of the leaf primordia. The AS1 promoter was selected based on its known specific expression pattern and the function of AS1 in leaf primordia, Byrne et al. 2000, Nature, 408(6815) 967-971.

The AT2G37630 polypeptide was used in a blast search followed by a phylogenetic analysis of the identified putative homologous and orthologous genes. The putative orthologous gene in Populus trichocarpa is Potri.006G085900. The identified Eucalyptus grandis ortholog, accession number Eucgr.K03130, has a polypeptide sequence identity of 67% to AT2G37630 over 98% of the E. grandis sequence. Promoter analysis in Arabidopsis has shown that the promoter is approximately 2.7 kb. The promoter, in both Arabidopsis and Eucalyptus, contains a large intron in the predicted 5′ UTR. A 2700 base pair long promoter fragment immediately upstream of, but not including, the start codon of gene Eucgr.K03130 was selected for synthesis of the pEA3 promoter, Seq ID No: 5. Orthologous to the pEA3 promoter is the promoter region, pEA3-ort poplar, Seq ID No: 24, of the Populus trichocarpa gene with accession number Potri.006G085900.

The Promoter pEA4

The A. thaliana gene AT5G67260 (AtCYCD3:2) encode CYCD3;2, a CYCD3 D-type cyclin, which is important for determining cell number in developing lateral organs and mediating cytokinin effects in apical growth and development. CYCD3 function contributes to the control of cell number in developing leaves by regulating the duration of the mitotic phase and timing of the transition to endocycles. CYCD3;1 expression is restricted to the shoot apical meristem (SAM), very young primordia, and young hydathodes, whereas CYCD3;2 and CYCD3;3 reporters are also active in older leaf primordia, with CYCD3;2 expression persisting longest in young leaves. The phytohormone cytokinin regulates cell division in the shoot meristem and developing leaves and induces CYCD3 expression. Loss of CYCD3 impairs shoot meristem function and leads to reduced cytokinin responses, Dewitte et al., 2007 PNAS, 104(36) 14537-14542.

The AT5G67260 polypeptide was used in a blast search followed by a phylogenetic analysis of the identified putative homologous and orthologous genes. The identified Eucalyptus grandis ortholog, accession number Eucgr.I00802, has a polypeptide sequence identity of 51% to AT5G67260 over 94% of the E. grandis sequence. In Populus trichocarpa two putative orthologous genes are identified, Potri.007G048300 and Potri.005G141900; these two genes are considered paralogous genes. Promoter analysis in Arabidopsis has shown that the promoter fragment is approximately 2.5 kb. Therefore, a 2500 base pair long promoter fragment immediately upstream of, but not including, the start codon of gene Eucgr.I00802 was selected for synthesis of the pEA4 promoter, Seq ID No: 6.

The putative orthologous promoters to the pEA4 promoter are the Populus trichocarpa promoter regions, pEA4-ort poplar, Seq ID No: 25, and pEA4-para poplar, Seq ID No: 29.

The Promoter pEC1

The WOX4 gene in A. thaliana is preferentially expressed in the procambial/cambial stem cells and is a regulator of vascular stem cell proliferation, Mizukami and Fischer (2000) PNAS, 97(2): 942-947. The expression pattern of the hybrid aspen ortholog (HB3/WOX4) was first identified in a high resolution expression profile over the vascular cambium, Schrader et al. 2004, The Plant Cell 16(9) 2278-2292, subsequently using more precise methods such as promoter:GUS analysis, real-time PCR and in-situ hybridization Nilsson, Doctoral thesis 2010:29 Faculty of Forest Sciences, Umea. These studies combined show that WOX4/HB3 is a cambium specific promoter well suited for tissue specific expression of chosen trait genes. The Eucalyptus gene Eucgr.F02320 forms a phylogenetic group with the Arabidopsis WOX4 (AT1G46480) and two P. trichocarpa homologs Potri.014G025300 and Potri.002G124100. Alignment of 4 kb fragments upstream of the coding sequence of the hybrid aspen transcripts with 4 kb upstream of the Eucgr.F02320 gene reveals major similarities of approximately 2.1 kb. This region was selected for synthesis of the stem/cambium specific promoter pEC1, Seq ID No: 7.

The putative orthologous promoters to the pEC1 promoter are the Populus trichocarpa promoter regions, pEC1-ort poplar, Seq ID No: 26, and pEC1-para poplar, Seq ID No: 30.

The Promoters pEL1.1 and pEL1.2

The pEL1.1 and pEL1.2 promoters originate from the one of the best characterized light-inducible genes in leaves, the small subunit of ribulose-1,5-bisphosphate carboxylase (RuBisCo or RBCS) gene promoter. The Rubisco small subunit, RBCS, is a multigene family in Arabidopsis thaliana and consists of four genes; RBCS1A (At1g67090), RBCS1B (At5g38430), RBCS2B (At5g38420), and RBCS3B (At5g38410).

It has been found that the promoter from RBCS genes contain an intricate assortment of positive and negative regulatory elements that are able to confer light-inducible and tissue-specific expression in transgenic plants (Gilmartin and Chua 1990, Mol Cell Biol, 10(10) 5565-5568). Anisimov et al. 2007, Mol Breeding, 19, 241-253, describes that the level of expression conferred by the pRBCS promoter differ depending on the length of the used promoter fragment. A longer promoter of 1.6 kb has an expression level that is four times higher than a short promoter fragment of 300-600 bp.

The four polypeptides of the RBCS multigene family from Arabidopsis thaliana were used in a blast search followed by a phylogenetic analysis of the identified putative homologous and orthologous genes.

The three identified loci, Eucgr.B03013, Eucgr.J01502, and Eucgr.K02223, were found to have 70-80% amino acid identity to query sequence. The highest scoring, Eucgr.B03013, has 79.7%, 80.2%, 80.2% and 79.1% identity, respectively, to the above-mentioned Arabidopsis thaliana genes. In the phylogenetic analysis the Eucgr.K02223 gene was identified as the closest homologue to Arabidopsis thaliana RBCS. In Populus trichocarpa two putative orthologous genes are identified, Potri.017G114600 and Potri.004G100000, these two genes are considered paralogous genes.

Based on the findings of Anisimov, et al. (2007). Mol Breeding, 19, 241-253, two promoter fragments of different lengths were selected for synthesis; pEL1.1, Seq ID No: 8, has a short promoter sequence of 600 bp, while pEL1.2, Seq ID No: 9, has a longer promoter sequence of 1800 bp.

The putative orthologous promoters to the pEL1.1 promoter are the Populus trichocarpa promoter regions, pEL1.1-ort poplar, Seq ID No: 27, and pEL1.1-para poplar, Seq ID No: 31. The putative orthologous promoters to the pEL1.2 promoter are the Populus trichocarpa promoter regions, pEL1.2-ort poplar, Seq ID No: 28, and pEL1.2-para poplar, Seq ID No: 32.

Promoter from Hybrid Aspen

The gene AINTEGUMENTALIKE1 (AIL1), Potri.002G114800, is expressed in meristems during active growth, while its activity is down-regulated under day length shortening, Karlberg et al., 2011, PLoS Genet. November; 7(11):e1002361). The promoter was cloned as shown in the experimental part below. The pAIL1 promoter consists of a 2683 base pair long fragment, Seq ID No: 10.

The pEA2 and pAIL1 are orthologous promoters.

### Functional Tests of the Identified Promoters.

In order to verify that all newly identified Eucalyptus promoters including the two variants of the leaf specific promoter were functional in trees, transgenic hybrid aspen with the different recombinant promoter-GUS constructs were created and studied. The DNA sequence of the identified promoter regions of the genomic sequence were manufactured by DNA synthesis, creating identical copies of the identified promoter regions of the genomic sequence of Eucalyptus grandis.

The synthetic promoters were cloned into an expression vector, positioned in front of the beta-glucuronidase (GUS) reporter gene. The recombinant promoter-GUS constructs were used in Agrobacterium-mediated transformation of hybrid aspen.

The promoter expression pattern was determined by histological studies of transgenic hybrid aspen plants harbouring the promoter-GUS construct, where the expression of the GUS gene was monitored using the established histochemical GUS staining technique. Details for these experiments are found in example 3

Eucalyptus promoters having a desired expression pattern could subsequently be used for controlling gene expression, to specifically direct the expression of a trait gene in planta.

### Gibberellin Genes

Variants of the GA 20-oxidase gene from Eucalyptus, Arabidopsis and Populus were included in the development of combinations of promoters and GA 20-oxidase coding sequences.

A number of cell type specific and tissue-specific promoters expressed in actively growing tissues were used to direct GA 20-oxidase activity in the plant. The specificity of these promoters make them ideal for affecting actively growing cells while minimizing side effects on cells not actively involved in growth.

TABLE 2

| | Amino acid | | Nucleotide | |
|---|---|---|---|---|
| | sequence | Number of | sequence | Number of |
| Gene name | Seq ID No: | amino acids | Seq ID No: | Nucleotides |
| AtGA20ox1 | 12 | 377 | 13 | 1134 |
| AtGA20ox3 | 14 | 380 | 15 | 1143 |
| PttGA20ox1 | 16 | 385 | 17 | 1158 |
| EucGA20ox1 | 18 | 385 | 19 | 1158 |

A GA20ox gene useful in the present invention is a nucleic acid encoding a gibberellin 20-oxidase gene product that shows gibberellin 20-oxidase activity and preferably has an amino acid sequence at least 50%, such as 60%, 70%, 75%, 80%, 85%, 90%, 95%, 99% or 100% identical to an amino acid sequence having the amino acid sequence according to SEQ ID NO: 16 (PttGA20ox1) or SEQ ID NO: 18 (EucGA20ox1) in corresponding assays.

### Plant Transformation

DNA constructs were transformed into Agrobacterium and subsequently into hybrid aspen, where Populus tremula x tremuloides clone T89, also called “poplar” in this application, was transformed and regenerated. Typically, 8 independent lines were generated for each construct. One such group of transgenic trees produced using the same DNA construct is hereafter called a “construction group”, that is different transgenic trees emanating from one construct.

Each transgenic line within each construction group derives from a different transformation event and has most probably the recombinant DNA inserted into a unique location in the plant genome. This makes the different transgenic lines within one construction group partly different. For example it is known that different transformation events will produce plants with different expression levels of the gene product. It is also known that different levels of expression of a gene will result in different levels of phenotypic effects.

### Plant Growth

The transgenic hybrid aspen lines were grown together with wild type control (wt) trees, in a greenhouse under a photoperiod of 18 h and a temperature of 22° C./15° C. (day/night). All transgenic lines were grown in three clonal replicates. The plants were grown for 8-9 weeks before harvest and fertilized weekly. During this time height and diameter were measured weekly. Wild type (typically 35-45 trees) and transgenic trees were grown in parallel in the greenhouse under the same conditions. All comparisons between wild type trees and the transgenic trees with a specific promoter-gene combination are made within the cultivation group.

### Growth Analyses

To identify construction groups showing a significant difference compared to the wild type population, data from each construction group was subjected to a number of growth data analyses of growth/biomass and wood density measurements.

After 8 to 9 weeks growth in the greenhouse the trees were harvested and sampled. Two principal types of harvests were used; either a general setup designed for e.g. chemical analysis, wood morphology analysis, gene expression analysis, wood density analysis and metabolomics analysis, or a second setup designed for dry weight measurements of bark, wood, leaves and roots.

Measurements of plant height and diameter were recorded one to two times per week during the cultivation and before harvest of the plants. Final height and diameter measurements were subsequently used to identify construction groups with altered growth characteristics.

The volume of the stem of each individual plant was approximated from final height and final diameter measurements using the formula for volume of a cone.

Stem volume approximation:

$$V = \frac{{π}^{*} {r}^{2 *} h}{3}$$

where: V=Volume; h=height (Final height), r=radius (Final diameter/2)

Average final volumes of each construction group population and corresponding wild type population were subsequently calculated.

### Wood Density Analyses

Wood density is an important trait for increasing biomass production. An increase in wood density increases the energy content per cubic metre reduces the volume of a fixed amount of biomass and hence, e.g. the volume required to transport a fixed amount of biomass. Correspondingly, more biomass can be transported per volume. Therefore increased density is of interest, even if total biomass is not increased. Increased density could also be of benefit coupled to pulp and paper production.

A 5 cm long stem segment, sampled between 36 and 41 cm from the soil from each harvested plant and stored in a freezer after harvest, was used for density measurements. Samples to be analysed were thawed followed by removal of bark and pith. The weight (w) was measured using a balance and the volume (V) was determined using the principle of Archimedes, where wood samples were submerged (using a needle) into a beaker (placed on a balance) with water. The recorded increase in weight is equivalent to the weight of the water displaced by the wood sample. Since the density of water is 1 g/cm3 at ambient room temperature the recorded increase is also equivalent to the volume of the wood sample. The samples were subsequently dried in oven for >48 h at 60° C.

The dry weights (dw) were measured and the density (d) was calculated according to:

$$d = \frac{d w}{V}$$

Samples from each construction group were compared to wild type samples from the same cultivation.

### Analysis of Expression Levels

Real-time RT-PCR was used to compare construct gene expression levels of the construction group with corresponding wild type group. The expression level of 26S proteasome regulatory subunit S2 was used as a reference to which construct gene expression was normalized. The comparative CT method was used for calculation of relative construct gene expression level, where the ratio between construction and reference gene expression level is described by (1+E_{target})−CT_{target}/(1+E_{reference})−CT_{reference}, where E_{target} and E_{reference} are the efficiencies of construct and reference gene PCR amplification respectively and CT_{target} and CT_{reference} are the threshold cycles as calculated for construct and reference gene amplification respectively.

### Obtaining Plants

The present invention extends to any plant cell of the above genetically modified, or transgenic plants obtained by the methods described herein, and to all plant parts, including harvestable parts of a plant, seeds, somatic embryos and propagules thereof, and plant explant or plant tissue. The present invention also encompasses a plant, a part thereof, a plant cell or a plant progeny comprising a DNA construct according to the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. It should be noted that embodiments and features described in the context of one of the aspects of the present invention also apply to the other aspects of the invention. Thus, definitions of one embodiment regard mutatis mutandis to all other embodiments comprising or relating to the one embodiment. When for example definitions are made regarding DNA constructs or sequences, such definitions also apply with respect to methods for producing a plant, vectors, plant cells, plants comprising the DNA construct and vice versa. A DNA construct described in relation to a plant also regards all other embodiments.

### Methods for Enhancing the Productivity of a Plant by Genetic Modification

One or more of the constructs according to the invention may be introduced into a plant cell by transformation.

Transformation of Plant Cells

In accordance with the present invention, the method comprises transforming regenerable cells of a plant with a nucleic acid construct or recombinant DNA construct (as described in I) and regenerating a transgenic plant from said transformed cell. Production of stable, fertile transgenic plants is now a routine method.

Various methods are known for transporting the construct into a cell to be transformed. Agrobacterium-mediated transformation is widely used by those skilled in the art to transform tree species, in particular hardwood species such as poplar and Eucalyptus. Other methods, such as microprojectile or particle bombardment, electroporation, microinjection, direct DNA uptake, liposome mediated DNA uptake, or the vortexing method may be used where Agrobacterium transformation is inefficient or ineffective, for example in some gymnosperm species.

A person of skill in the art will realize that a wide variety of host cells may be employed as recipients for the DNA constructs and vectors according to the invention. Non-limiting examples of host cells include cells in embryonic tissue, callus tissue type I, II, and III, hypocotyls, meristem, root tissue, tissues for expression in phloem, leaf discs, petioles and stem internodes. Once the DNA construct or vector is within the cell, integration into the endogenous genome can occur.

Selection of Transformed Plant Cells and Regeneration of Plant or Woody Plants

Following transformation, transgenic plants are preferably selected using a dominant selectable marker incorporated into the transformation vector. Typically, such a marker will confer antibiotic or herbicide resistance on the transformed plants and selection of transformants can be accomplished by exposing the plants to appropriate concentrations of the antibiotic or herbicide. A selection marker using the D-form of amino acids and based on the fact that plants can only tolerate the L-form offers a fast, efficient and environmentally friendly selection system.

Subsequently, a plant may be regenerated, e.g. from single cells, callus tissue or leaf discs, as is standard in the art. Almost any plant can be entirely regenerated from cells, tissues and organs of the plant. After transformed plants are selected and they are grown to maturity and those plants showing altered growth properties phenotype are identified.

### Methods for Detecting Modified Expression of a Gene Encoding a Polypeptide in a Plant or Woody Plant of the Invention

Real-time RT-PCR can be used to compare gene expression, i.e. the mRNA expression levels, in a genetically modified (GM) plant or woody plant with the corresponding non-GM plant or woody plant. The amount of the polynucleotides disclosed herein can be determined using Northern blots, sequencing, RT-PCR or microarrays.

Western blots with immune detection or gel shift assays can be used to measure the expression levels or amounts of a polypeptide expressed in a GM plant or woody plant of the invention. Antibodies raised to the respective polypeptide may be used for specific immune-detection of the expressed polypeptide in tissue derived from a woody plant.

Eucalyptus plants are generated in a similar way, through transformation, regeneration and growth analysis.

The invention is further illustrated below by way of examples. The examples are not intended to restrict the scope of the invention, which is that of the appended claims.

### EXAMPLES

### Example 1 Constitutive Expression May have Disadvantageous Effects

Overexpression of a gene may elicit different phenotypical effects under two distinctly different growth conditions. In WO2009084999, the recombinant DNA construct, named PttTF0002, was used to constitutively over-express a trait gene in hybrid aspen trees using the CaMV 35S promoter, which resulted in a growth rate increase of 36% compared to wild type when grown in greenhouse.

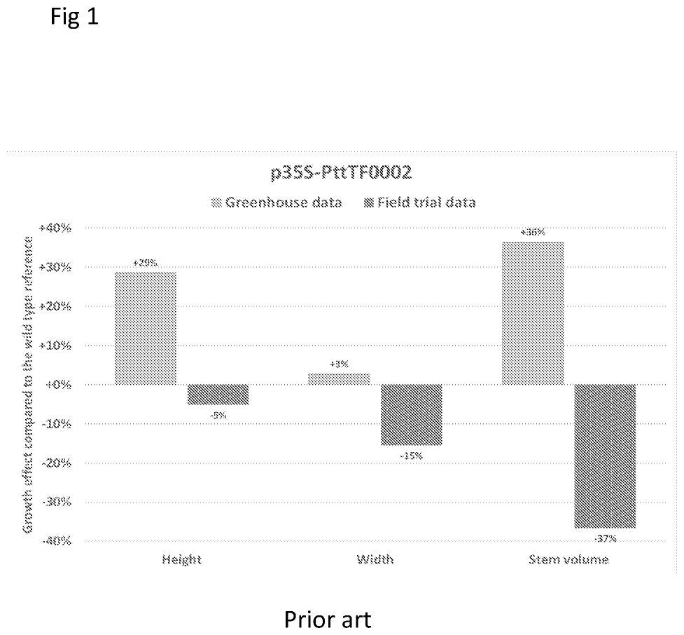

The same hybrid aspen trees were planted in an open field during spring of 2010 in the county of Halland, located on the southern west coast of Sweden. However, it was discovered that the transgenic trees harbouring the 35S promoter construct PttTF0002 did not perform as well as expected in these field growth tests. The observed average stem volume increase of 36% in greenhouse tests was contrasted with a considerable growth reduction of 37% compared to the wild type reference trees grown at the same test site location, FIG. 1. This result was much unexpected, since the 35S promoter construct consistently worked very well under greenhouse conditions.

### Example 2 Cloning of Promoters

### Cloning of Eucalyptus Promoters:

The identification of novel Eucalyptus promoters is described in the detailed description above. All Eucalyptus promoters were cloned in the same way. The promoter DNA fragments were manufactured by DNA synthesis, using the DNA sequences of the identified promoter regions of the publically available Eucalyptus grandis genome as a template, thus creating identical copies of the corresponding Eucalyptus grandis promoter regions. The synthesized promoter fragments were flanked by Gateway recombination sites for sub-cloning purposes. All promoter fragments were sub-cloned using Gateway recombination into the pK7m24GW,3 vector (VIB, Rijvisschestraat 120, B-9052 Zwijnaarde, Belgium), where they were placed upstream of and thus controlling the expression of a trait gene. The novel combinations of promoters and genes are further described in Example 4, below. In the same way, for promoter expression studies, the promoters were all sub-cloned using Gateway recombination into the pK7m24GW,3 vector and placed in front of the beta-glucuronidase (GUS) reporter gene.

2.1 the Constitutive Promoter pECO1

The DNA sequence upstream of the Eucalyptus grandis gene with accession Eucgr.E00053 was thoroughly investigated as described (in the detailed description) above. A fragment of 1084 nucleotides immediately upstream, but not including, the start codon was selected to define the pECO1 promoter, Seq ID No: 1.

2.2 the Constitutive Promoter pECO2

The DNA sequence upstream of the Eucalyptus grandis gene with accession Eucgr.H04673 was thoroughly investigated as described (in the detailed description) above. A fragment of 2000 nucleotides immediately upstream, but not including, the start codon was selected to define the pECO2 promoter, Seq ID No: 2.

2.3 the Tissue-Specific Promoter pEA1

The DNA sequence upstream of the Eucalyptus grandis gene with accession Eucgr.C00732 was thoroughly investigated as described (in the detailed description) above. A fragment of 2000 nucleotides immediately upstream, but not including, the start codon was selected to define the pEA1 promoter, Seq ID No: 3.

2.4 the Tissue-Specific Promoter pEA2

The DNA sequence upstream of the Eucalyptus grandis gene with accession Eucgr.F02223 was thoroughly investigated as described (in the detailed description) above. A fragment of 2500 nucleotides immediately upstream, but not including, the start codon was selected to define the pEA2 promoter, Seq ID No: 4.

2.5 the Tissue-Specific Promoter pEA3

The DNA sequence upstream of the Eucalyptus grandis gene with accession Eucgr.K03130 was thoroughly investigated as described (in the detailed description) above. A fragment of 2700 nucleotides immediately upstream, but not including, the start codon was selected to define the pEA3 promoter, Seq ID No: 5.

2.6 the Tissue-Specific Promoter pEA4

The DNA sequence upstream of the Eucalyptus grandis gene with accession Eucgr.I00802 was thoroughly investigated as described (in the detailed description) above. A fragment of 2500 nucleotides immediately upstream, but not including, the start codon was selected to define the pEA4 promoter, Seq ID No: 6.

2.7 the Tissue-Specific Promoter pEC1

The DNA sequence upstream of the Eucalyptus grandis gene with accession Eucgr.F02320 was thoroughly investigated as described (in the detailed description) above. A fragment of 2101 nucleotides immediately upstream, but not including, the start codon was selected to define the pEC1 promoter, Seq ID No: 7.

2.8 the Tissue-Specific Promoters, pEL1.1 and pEL1.2

The DNA sequence upstream of the Eucalyptus grandis gene with accession Eucgr.K02223 was thoroughly investigated as described (in the detailed description) above. Based on these studies two promoter variants were selected; a shorter and a longer promoter fragment. Fragments of 600 and 1800 nucleotides immediately upstream, but not including, the start codon were selected to define the shorter pEL1.1 (Seq ID No: 8) and longer pEL1.2 promoter variants respectively (Seq ID No: 9).

Cloning of the Tissue-Specific Hybrid Aspen Promoter pAIL1

The gene AINTEGUMENTALIKE1 (AIL1), Potri.002G114800, is expressed in meristems during active growth, while its activity is down-regulated under day length shortening. (Karlberg et al., 2011, PLoS Genet. November; 7(11):e1002361)

The promoter region was amplified by PCR from the pENTR ANT promoter construct, which carries the genomic DNA from the hybrid aspen promoter sequence of AIL1, using AIL1 promoter sequence specific primers flanked by Sac I and Spe I restriction sites to facilitate further cloning; pAIL1-Forward (the Sac I site underlined) 5′-GCAGAGCTCGGGGAATGATAGGCTGACAAG-3′, Seq ID No: 33, and pAIL1-Reverse (Spe I site underlined) 5′-GCAACTAGTCCCAAAATCTTGCCTACTTCCAT, Seq ID No: 34. The amplified PCR fragment was after digesting with Sac I and Spe I used for further generation of vector constructs aimed for transformation of plants.

The resulting AIL1 promoter, pAIL1, consist of a 2683 base pair long fragment excluding the restriction sites used for cloning, Seq ID No: 10.

### Example 3 Verification of Expression Pattern of the Eucalyptus Promoters

The expression patterns of the Eucalyptus promoters were determined by histological studies of transgenic hybrid aspen plants harbouring the promoter-GUS construct. Promoter activity was assayed using the established histochemical GUS staining technique.

Samples were collected from young transgenic plants. Five to eight transgenic lines from each promoter-GUS construct were sampled and the following eight parts of the plant were stained for GUS expression; 1) Apex with leaf primordia and small young leaf; 2) Part of young leaf; 3) Young stem section, close to apex; 4) Part of petiole; 5) Axillary bud; 6) Part of old leaf; 7) Longitudinal stem section of old stem and 8) Root. The stained plant tissues were carefully studied under a light microscope.

### Results:

The resolution of the GUS assay is sufficient to distinguish the tissue regions from which the product of GUS enzyme activity emanates, but not high enough to distinguish the specific cells from which the product of GUS enzyme activity emanates.

pEA1: Tissue-specific expression in the regions of the meristematic tissue responsible for primary growth in the apex, axillary buds and in leaf primordia was confirmed.

pEA2: Tissue-specific expression in the regions of the actively dividing cells of the apex, in axillary buds and in the vascular tissues of young and older stem was confirmed.

pEA3: Very faint tissue-specific expression in the regions of the meristematic tissues responsible for primary growth in the apex and axillary buds was confirmed.

pEA4: Weak tissue-specific expression in the regions of meristematic tissues responsible for primary and secondary growth in the apex, cambium and root was confirmed.

pEC1: Expression in the vascular tissues of young stem, older stem, root and leaf as well as in root tip. However, the resolution of the GUS assay is not high enough to distinguish the specific cells of the vascular tissue from which the product of GUS enzyme activity emanates.

pECO1: Constitutive expression was confirmed in early stages of transgenic tissue formation. Faint expression observed in older plant tissues.

pECO2: Strong constitutive expression was confirmed.

pEL1.1: Strong green-tissue-specific expression, also in light-exposed root tissues was confirmed.

pEL1.2: Strong green-tissue-specific expression, also in light-exposed root tissues was confirmed.

### Example 4: Construction of Novel Promoter-Gene Combinations

As described in Example 2 the Eucalyptus promoter DNA fragments were manufactured by DNA synthesis and flanked by Gateway recombination sites for sub-cloning purposes. All promoter fragments were sub-cloned using Gateway recombination into the pK7m24GW,3 vector, where they were placed upstream of and thus controlling the expression of a GA 20-oxidase gene in combinations of promoter and gene as described below.

### Construct F130

The GA 20-oxidase gene 1 from Arabidopsis thaliana, Seq ID No: 13, was operably linked with the pEA1 promoter, Seq ID No: 3, to create the recombinant DNA construct F130, pEA1-AtGA20ox1. This construct can be used to increase GA 20-oxidase levels specifically in the shoot apical meristem and organ primordia.

### Construct F140

In order to test if different origins of the GA 20-oxidase gene 1 may influence the phenotype, the GA 20-oxidase gene 1 from Populus tremula x tremuloides, Seq ID No: 17, was operably linked with the pEA1 promoter, Seq ID No: 3, to create the recombinant DNA construct F140, pEA1-PttGA20ox1. This construct can be used to increase GA 20-oxidase levels specifically in the shoot apical meristem and organ primordia.

### Construct F131

The GA 20-oxidase gene 1 from Arabidopsis thaliana, Seq ID No: 13, was operably linked with the pEA2 promoter, Seq ID No: 4, to create the recombinant DNA construct F131, pEA2-AtGA20ox1. This construct can be used to increase GA 20-oxidase levels specifically in the actively dividing cells in the cambial region of the stem and the shoot apical meristem.

### Construct F132

The GA 20-oxidase gene 1 from Arabidopsis thaliana, Seq ID No: 13, was operably linked with the pEA3 promoter, Seq ID No: 5, to create the recombinant DNA construct F132, pEA3-AtGA20ox1. This construct can be used to increase GA 20-oxidase levels specifically in the leaf forming tissues of the leaf primordia.

### Construct F133

The GA 20-oxidase gene 1 from Arabidopsis thaliana, Seq ID No: 13, was operably linked with the pEA4 promoter, Seq ID No: 6, to create the recombinant DNA construct F133, pEA4-AtGA20ox1. This construct can be used to increase GA 20-oxidase levels specifically in the shoot apical meristem, leaf primordia and to some extent in younger leaves.

### Construct F134

Furthermore, the GA 20-oxidase gene 1 from Arabidopsis thaliana, Seq ID No: 13, was operably linked with the pEC1 promoter, Seq ID No: 7, to create the recombinant DNA construct F134, pEC1-AtGA20ox1. This construct can be used to increase GA 20-oxidase levels specifically in the procambial/cambial stem cells.

### Construct F135 and F136

The GA 20-oxidase gene 1 from Arabidopsis thaliana, Seq ID No: 13, was operably linked with the pEL1.1 promoter, Seq ID No: 8, to create the recombinant DNA construct F135 and with the pEL1.2 promoter, Seq ID No: 9, to create the recombinant DNA construct F136. These two constructs can be used to strongly increase GA 20-oxidase levels in all green tissues of the plant.

### Construct F128

The GA 20-oxidase gene 1 from Arabidopsis thaliana, Seq ID No: 13, was operably linked with the weak constitutive pECO1 promoter, Seq ID No: 1, to create the recombinant DNA construct F128, pECO1-AtGA20ox1. This construct can be used to increase GA 20-oxidase levels in all tissues of the plant.

### Construct F139

The GA 20-oxidase gene 1 from Populus tremula x tremuloides, Seq ID No: 17, was combined with the weak constitutive pECO1 promoter, Seq ID No: 1, to create the recombinant DNA construct F139, pECO1-PttGA20ox1. This construct can be used to increase GA 20-oxidase levels in all tissues of the plant.

### Construct F129

The GA 20-oxidase gene 1 from Arabidopsis thaliana, Seq ID No: 13, was operably linked with the strong constitutive pECO2 promoter, Seq ID No: 2, to create the recombinant DNA construct F129, pECO2-AtGA20ox1. This construct can be used to strongly increase GA 20-oxidase levels in all tissues of the plant.

### Construct F127 and F137

The strong constitutive promoter p35S was operably linked with two different Arabidopsis thaliana GA 20-oxidase genes, GA 20-oxidase 1, Seq ID No: 13, and GA 20-oxidase 3, Seq ID No: 15, creating the recombinant DNA construct F127, p35S-AtGA20ox1, and the recombinant DNA construct F137, p35S-AtGA20ox3, respectively. These two constructs can be used to strongly increase GA 20-oxidase levels in all tissues of the plant.

### Construct F138

The strong constitutive promoter p35S was operably linked with the GA 20-oxidase gene 1 from Populus tremula x tremuloides, Seq ID No: 17, to create the recombinant DNA construct F138, pECO1-PttGA20ox1. This construct can be used to strongly increase GA 20-oxidase levels in all tissues of the plant.

### Construct F141

Furthermore, the strong constitutive promoter p35S was operably linked with the GA 20-oxidase gene 1 from Eucalyptus grandis x urophylla, Seq ID No: 19, to create the recombinant DNA construct F141, p35S-EucGA20ox1. This construct can be used to strongly increase GA 20-oxidase levels in all tissues of the plant.

Construct pAIL1:GA20ox1

The AIL1 promoter fragment, Seq ID No: 10, was ligated into the Gateway vector pK2GW7,0 generating an AIL1 promoter containing pK2GW7,0 vector (pK2GW7-pAIL1). Thereafter the coding sequence of Arabidopsis GA 20-ox1 (AtGA20-ox1) was amplified by PCR using a previously cloned coding part of the Arabidopsis GA 20-ox1 gene as template and primers including the Gateway recombination sites and gene specific sequences; AtGA20ox.1-attB1 Forward 5′-GGGACAAGTTTGTACAAAAAAGCAGGCTTAATGGCCGTAAGTTTCGTAACAAC-3′ Seq ID No: 35 and AtGA20ox.1-attB2 Reverse 5-GGGGACCACTTTGTACAAGAAAGCTGGGTCTTAGATGGGTTTGGTGAGCCAAT-3′, Seq ID No: 36.

The fragment was subsequently cloned into pDONR207 by a BP recombination reaction and this ‘Entry clone’ was used in an LR recombination reaction in order to introduce the coding sequence of AtGA20-ox1, Seq ID No: 13, in to pK2GW7-pAIL1 using the Gateway technology creating an ‘Expression clone’ pAIL1:GA20ox1. The final expression clone then contained the promoter, pAIL1, the recombination site, attR1, the AtGA20ox1 coding sequence, the recombination site, attR2 and the transcription terminator from CaMV 35S, in said order.

This construct can be used to increase GA 20-oxidase levels specifically in the actively dividing cells in the cambial region of the stem and the shoot apical meristem.

### Example 5 Transformation of Hybrid Aspen

The DNA constructs described in Example 4 were transformed into hybrid aspen (Populus tremula x Populus tremuloides Michx., clone T89) by Agrobacterium-mediated transformation. The transformation and regeneration of transgenic plants were performed as described in the experimental part of WO2016108750. Typically, 8 independent transgenic lines were generated for each construct.

### Example 6.1 Hybrid Aspen Grown and Growth Analysis from Greenhouse

For each promoter-gene construct, three transgenic hybrid aspen lines in three clonal replicates each were grown together with wild type reference trees in the greenhouse, as described in the experimental part of WO2016108750 and in the detailed description above.

After 8 weeks of growing in the greenhouse the hybrid aspen trees were measured, harvested and sampled for the following traits, plant height, width, stem volume, average internode length and wood density.

Statistical analysis was used to determine phenotypical differences between transgenic and wild type trees. The population of transgenic trees from each promoter-gene combination was compared to the wild type population of trees with the Student's t-test and a stringent p-value cutoff of 0.01. Similarly, to identify the best performing transgenic lines, the population of trees from each transgenic line, that is, the three replicates, was compared to the wild type population of trees with the same statistical test and settings. The results of the statistical analyses are presented in tables 3-7 as the percentage differences between averages of the compared populations of transgenic and wild type trees, wild type being the reference point. Percentage differences that are statistically significant according to the statistical criteria specified above are marked with an asterisk (*) in tables 3-7.

### Results:

TABLE 3

| Con- | | | | | Stem | Stem dry | Bark dry | Internode | Wood |
|---|---|---|---|---|---|---|---|---|---|
| struct | Gene | Promoter | Height | Width | volume | weight | weight | length | density |
| F134 | AtGA20ox1 | pEC1 | 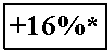 | +10% | 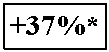 | 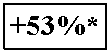 | 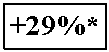 | 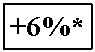 | 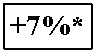 |
| F130 | AtGA20ox1 | pEA1 | +0% | −3% | −8% | −8% | −3% | 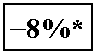 | +0% |
| F140 | PttGA20ox1 | pEA1 | +5% | +8% | +22% | +22% | +16% | −6% | +1% |
| F131 | AtGA20ox1 | pEA2 | +3% | +7% | +20% | +20% | +10% | 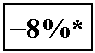 | +4% |
| F132 | AtGA20ox1 | pEA3 | 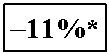 | −3% | −14% | −4% | −11% | 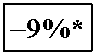 | 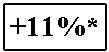 |
| F133 | AtGA20ox1 | pEA4 | −6% | +7% | +9% | +19% | +8% | 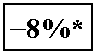 | +3% |

Significant differences (p < 0.01) compared to wild type marked with an asterisk * and shown in bold and in boxes.

TABLE 4

| | | | | | Stem | Stem dry | Bark dry | Internode | Wood |
|---|---|---|---|---|---|---|---|---|---|
| Construct | Gene | Promoter | Height | Width | volume | weight | weight | length | density |
| F134-Line1 | AtGA20ox1 | pEC1 | 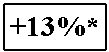 | +7% | +26% | +40% | +24% | +8% | +6% |
| F134-Line2 | | | 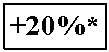 | +7% | +36% | 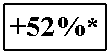 | +34% | +1% | +7% |
| F134-Line3 | | | 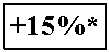 | +16% | 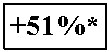 | 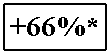 | +31% | +9% | +7% |
| F130-Line1 | AtGA20ox1 | pEA1 | −3% | −5% | −14% | −16% | −8% | −9% | +4% |
| F130-Line2 | | | +3% | −1% | −1% | −6% | +2% | 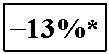 | +4% |
| F130-Line3 | | | +1% | −4% | −8% | −2% | −5% | −1% | −6% |
| F140-Line1 | PttGA20ox1 | pEA1 | +5% | +12% | +30% | +36% | +18% | +0% | −0% |
| F140-Line2 | | | +8% | +7% | +20% | +10% | +14% | 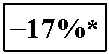 | +5% |
| F140-Line3 | | | +3% | +6% | +16% | +19% | +16% | −1% | −1% |
| F131-Line1 | AtGA20ox1 | pEA2 | +2% | +7% | +14% | +2% | +10% | 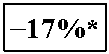 | +1% |
| F131-Line2 | | | −0% | −4% | −3% | +5% | −10% | −7% | 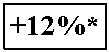 |
| F131-Line3 | | | +8% | 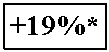 | 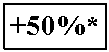 | 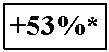 | +30% | −1% | +0% |
| F132-Line1 | AtGA20ox1 | pEA3 | −5% | −2% | −9% | +5% | −9% | −5% | 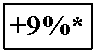 |
| F132-Line2 | | | 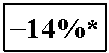 | +0% | −11% | −11% | −15% | −8% | 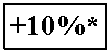 |
| F132-Line3 | | | 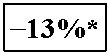 | −7% | −23% | −5% | −7% | 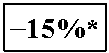 | 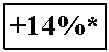 |
| F133-Line1 | AtGA20ox1 | pEA4 | −1% | +15% | +30% | +47% | +35% | −8% | −0% |
| F133-Line2 | | | −7% | +6% | +5% | +11% | −6% | −9% | −1% |
| F133-Line3 | | | −10% | −1% | −8% | +1% | −5% | −8% | 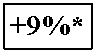 |

The results from each transgenic line presented individually. Significant differences (p < 0.01) compared to wild type marked with an asterisk (*) and shown in bold and in boxes.

By using a number of tissue-specific promoters to control the expression of the GA 20-oxidase gene, the inventors are able to demonstrate that specific over-expression of GA 20-oxidase does not necessarily lead to an increase in plant growth or they give different levels of growth increase. Conversely, specific over-expression of GA 20-oxidase will not generally have a large effect on plant growth. This demonstrates the non-obvious and inventive use of the specific combinations of promoters and genes disclosed herein to increase plant growth.

### Cambium-Specific Expression of GA 20-Oxidase Results in Increased Growth:

Constitutive over-expression of GA 20-oxidase is known to potentially cause adverse effects on rooting and to increase the risk of gene silencing. This risk of adverse effects can be reduced by using for example the pEC1 promoter, active specifically in the cambial region of the stem, to over-express GA 20-oxidase.

In plants where GA 20-oxidase is over-expressed using the pEC1 promoter growth is significantly improved compared to wild type; looking at the average of all tested transgenic lines, including the three replicates of each, the increase in plant height is 16%; average internode lengths increase by 6%; average stem diameter and stem volume increase by 10% and 37% respectively. Further, a substantial increase in stem and bark dry weights, of in average 53% and 29% respectively, is observed, as well as an increase in wood density of 7% in average. Dry weight and wood density results confirm that the increase in growth also includes a considerable increase in biomass production in the transgenic trees compared to wild type. If instead each transgenic line, including its three replicates, is compared to the wild type reference, remarkable increases in stem volume and stem dry weight by 51% and 66% respectively is observed in the most improved line; the increase in plant height is 15% in the same line. No adverse effects of GA 20-oxidase over-expression are observed.

Tissue-specific GA 20-oxidase over-expression provides a more efficient use of resources for the tree compared to constitutive over-expression. When the GA 20-oxidase gene is strongly over-expressed in the majority of cells throughout the plant using, for example, the 35S promoter constructs, large quantities of GA 20-oxidase enzyme are produced also in cells and tissues where there is little or no substrate to process. The impact on plant growth relative to the total amount GA 20-oxidase enzyme produced is therefore higher in transgenic plants with a tissue-specific GA 20-oxidase gene over-expression driven by, for example, the pEC1 promoter than in the 35S over-expressing plants. At the same time, tissue specific over-expression will reduce the risk of adverse effects that have been observed when GA 20-oxidase is over-expressed constitutively at high levels.

The group of cell proliferation/cell division associated promoters pEA1, pEA2 and pEA4 are all active in the regions of primary growth in the plant. The pEA3 promoter is expressed in the apical region of the plant, more specifically in the leaf forming tissues of the leaf primordia. No statistically significant positive phenotypical effect is observed when over-expressing GA 20-oxidase using the pEA1 or pEA4 promoters.

When the pEA3 promoter is used to over-express GA 20-oxidase, an increase in wood density as well as a reduction in plant growth compared to wild type is observed.

The pEA2 promoter, however, is also active in the vascular cambium of the stem. When pEA2 is used to over-express GA 20-oxidase, the best performing transgenic line has a significantly improved growth phenotype compared to wild type, with an average increase in stem diameter and stem volume of 19% and 50% respectively, cf. Table 4. A substantial increase in stem dry weight of 53% is also observed which confirms that biomass production is also significantly increased. This was again observed when tested in a field trial experiment, described in detail in Example 7.

Overexpressing the GA 20-oxidase gene with different tissue-specific promoters have different phenotypical effects which can be used to tailor the expression pattern of the gene to the specific growth condition at hand and to retain or further improve the positive phenotypical traits provided by the gene when growth conditions change.

### Constitutive Over-Expression

TABLE 5

| | | | | | Stem | Stem dry | Bark dry | Internode | Wood |
|---|---|---|---|---|---|---|---|---|---|
| Construct | Gene | Promoter | Height | Width | volume | weight | weight | length | density |
| F128 | AtGA20ox1 | pECO1 | +3% | +8% | +18% | +15% | +5% | 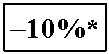 | +1% |
| F139 | PttGA20ox1 | pECO1 | +2% | +3% | +7% | −2% | +6% | +0% | +2% |
| F129 | AtGA20ox1 | pECO2 | 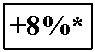 | +7% | +22% | +27% | +21% | −2% | −1% |

Significant differences (p < 0.01) compared to wild type marked with an asterisk * and shown in bold and in boxes.

The constitutive promoters pECO1 and pECO2 are both weaker than the 35S promoter. The level of gene over-expression conferred by the pECO1 promoter is too weak to significantly change the growth of the trees in this experiment. Internode length is slightly reduced when using pECO1 to drive the expression of the GA 20-oxidase gene from Arabidopsis.

TABLE 6

| | | | | | Stem | Stem dry | Bark dry | Internode | Wood |
|---|---|---|---|---|---|---|---|---|---|
| Construct | Gene | Promoter | Height | Width | volume | weight | weight | length | density |
| F127 | AtGA20ox1 | p35S | 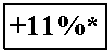 | 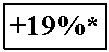 | 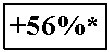 | 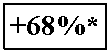 | 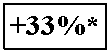 | 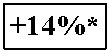 | +3% |
| F137 | AtGA20ox3 | p35S | 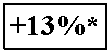 | 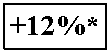 | 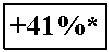 | 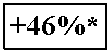 | +21% | 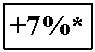 | +4% |
| F138 | PttGA20ox1 | p35S | 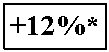 | 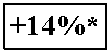 | 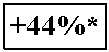 | 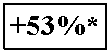 | 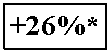 | 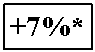 | 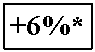 |
| F141 | EucGA20ox1 | p35S | 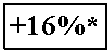 | 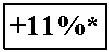 | 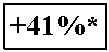 | 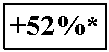 | +20% | 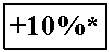 | +5% |

Significant differences (p < 0.01) compared to wild type marked with an asterisk * and shown in bold and in boxes.

Although known to potentially cause adverse effects on rooting and to increase the risk of gene silencing, constitutive over-expression was used to demonstrate the species conservation of the GA 20-oxidase gene and function and the strong positive effect that GA 20-oxidase over-expression can have on plant growth under controlled greenhouse conditions. Transgenic hybrid aspen trees harbouring a recombinant DNA construct, wherewith a GA 20-oxidase gene from either the Populus, Eucalyptus or Arabidopsis species is over-expressed using the strong constitutive 35S promoter, grow significantly faster, becoming taller, wider as well as having increased stem volume and dry weight compared to wild type trees. Independently of the origin of the GA 20-oxidase, the observed growth effect is consistent and significant. These findings indicate that the mode of operation of the GA 20-oxidase gene product is highly conserved between species and throughout the plant kingdom, as Populus and Eucalyptus, for example, are woody plant species and Arabidopsis is a small annual flowering plant.

TABLE 7

| | | | | | Stem | Stem dry | Bark dry | Internode | Wood |
|---|---|---|---|---|---|---|---|---|---|
| Construct | Gene | Promoter | Height | Width | volume | weight | weight | length | density |
| F135 | AtGA20ox1 | pEL1.1 | 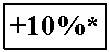 | +6% | +22% | +27% | 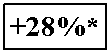 | −3% | +3% |
| F136 | AtGA20ox1 | pEL1.2 | 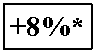 | 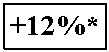 | 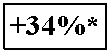 | 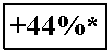 | 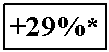 | −1% | +4% |

Significant differences (p < 0.01) compared to wild type marked with an asterisk * and shown in bold and in boxes.

The strong promoters pEL1.1 and pEL1.2 have an expression level and a broad pattern of expression in all green tissues of the plant that make them comparable to the 35S promoter. Thus, plants over-expressing GA 20-oxidase under either pEL1.1 or pEL1.2 promoter also results in an increased growth compared to wild type. Such a broad and strong expression could, however, potentially cause adverse effects and increase the risk of gene silencing similar to what has been observed with the 35S promoter.

### Example 6.2 Growth Effect Under Long and Short Days in pAIL1:GA20ox Hybrid Aspen

### Growth Conditions

Regenerated transgenic plants of pAIL1:GA20ox hybrid aspen and cuttings of wild type hybrid aspen plants, T89, were transferred to soil of fertilized peat. Plants were allowed to establish in soil for 3 weeks. They were fertilized once a week during growth in long day conditions. No fertilizer was added during short day conditions. Growth was done under long day (LD) conditions, i.e. 18 hours of light, 6 hours of dark, followed short day (SD) conditions, i.e. 15 hours of light, 9 hours of dark. The temperature during the light the dark period was constant 18° C. After bud set plants were grown under 8 hours of light, 16 hours of dark, at 6° C.±1° C.

### Measuring and Scoring

The height of plants were measured once a week starting 3 weeks after potting and grown in long day conditions and during subsequent day length shortening.

### Bud Set Scoring

Bud set of plants were scored once a week under SD and checked for changes under cold conditions. The scores of the bud set were defined as four developmental stages 3), growing, many young leaves at an apical region; 2) internode elongation halting and showing leaves of two internodes opposite each other; 1) apical bud with soft scales at a tip of the bud; 0) brownish apical bud, Ibáñez et al., 2010, Plant Physiol. August; 153:1823-33.

### Apical and Lateral Bud Burst Scoring

Bud burst of the plants were scored approximately twice a week using scoring as defined by Ibáñez et al., 2010, Plant Physiol. August; 153:1823-33.

The scores of the apical and lateral bud burst were defined as the six developmental stages 0) dormant bud; 1) swelling bud; 2) sprouting bud with the tips of the small leaves; 3) bud completely opened with leaves still clustered together; 4) leaves diverging with their blades still rolled up; 5) leaves completely unfolded.

### Growth and Diameter Measurement

Growth and diameter was measured as earlier presented.

### Results

Growth and diameter are summarized in Table 8.

It was surprisingly noted that positive growth effects were obtained with pAIL1:GA20ox1 under both long and short day growth conditions.

Measurements showed that the best line shows a 10% better height growth under 18/6 than wild type controls (T89), and they continue growing even better under 15/9 with 36% better height growth than wild type (T89). Taken together this supports that the pAIL1 promotor expressed in meristems promote increased growth.

In addition, all three lines (pAIL1:GA20ox1) showed a significantly delayed bud set but a wild type like bud burst phenotype. However compared to 35S:Ga20ox over expressors, these lines show bud set when the 35S over expressers show a completely impaired short day dormancy response. The promoter pAIL1 therefor also enables the use of Ga20ox over expression in a temperate climate with cold winters.

Like pEA2 and pEC1, the pAIL1 promoter is active in the meristematic cells giving rise to vascular tissue, such as the cambial region of the stem. Thus, the construct pAIL1:GA20ox1 can be used to increase GA 20-oxidase levels specifically in meristematic cells giving rise to vascular tissue, such as the cambial region of the stem. Like when using pEA2 or pEC1, an improved growth phenotype compared to wild type is observed when pAIL1 is used to over-express GA 20-oxidase.

TABLE 8
Growth of transgenic hybrid aspen with the construct
pAIL1:GA20ox1 versus wild type hybrid aspen under
short day (SD) and/or long day (LD) conditions

Height growth (cm ± SD)

| | 8 weeks in LD | 10 weeks in SD |
|---|---|---|
| pAIL1:GA20ox1-10 | 59.4 ± 3.99 | 140.78 ± 28.12 |
| pAIL1:GA20ox1-3 | 61.76 ± 3.95 | 148.06 ± 22.98 |
| pAIL1:GA20ox1-8 | 62.61 ± 2.07 | 149.71 ± 13.06 |
| Wild type T89 | 56.96 ± 2.19 | 109.75 ± 19.55 |
| | 8 weeks in LD | 8 weeks in SD |
| pAIL1:GA20ox1-10 | 59.4 ± 3.99 | 129.07 ± 18.64 |
| pAIL1:GA20ox1-3 | 61.76 ± 3.95 | 138.5 ± 18.90 |
| pAIL1:GA20ox1-8 | 62.61 ± 2.07 | 137.42 ± 9.68 |
| Wild type T89 | 56.96 ± 2.19 | 105.41 ± 13.63 |

| | Diameter growth (mm) |
|---|---|
| | 8 weeks in SD |
| pAIL1:GA20ox1-10 | 6.29 ± 1.46 |
| pAIL1:GA20ox1-3 | 6.68 ± 0.95 |
| pAIL1:GA20ox1-8 | 6.45 ± 0.61 |
| Wild type T89 | 5.10 ± 0.70 |
| | Number of internodes |
| | (after 10 weeks of SD) |
| pAIL1:GA20ox1-10 | 65.28 ± 13.19 |
| pAIL1:GA20ox1-3 | 68.00 ± 9.38 |
| pAIL1:GA20ox1-8 | 71.71 ± 5.28 |
| Wild type T89 | 56.00 ± 7.12 |

### Example 7a Hybrid Aspen Field Trial Experiments

The same transgenic hybrid aspen lines that were studied in the greenhouse experiment, described in detail in Example 6, were again propagated from tissue culture material for a field trial experiment. Wild type reference plants were propagated in parallel and treated exactly as the transgenic plants throughout the experiment. Plants were grown in vitro until ready for planting in soil. The plants were hardened during a period of five weeks; the first two weeks to establish rooting in soil in the greenhouse and then another three weeks in outdoor growth conditions. After this the plants were transported to the field site and kept in pots in outdoor conditions for 5 weeks before planting into the field. The height of the plants were measured at planting and used for statistical analysis.

Statistical analysis was used to determine phenotypical differences between transgenic and wild type trees. First, a General Linear Model ANOVA was performed on the entire dataset. Second, the population of transgenic trees from each promoter-gene combination was compared to the wild type population of trees using the established Dunnett's multiple comparison of means method and a stringent p-value cutoff of 0.01. Table 9 below.

TABLE 9

| Construct | Gene | Promoter | Height |
|---|---|---|---|
| F127 | AtGA20ox1 | p35S | 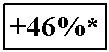 |
| F138 | PttGA20ox1 | p35S | 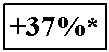 |
| F129 | AtGA20ox1 | pECO2 | 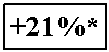 |
| F134 | AtGA20ox1 | pEC1 | 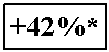 |
| F136 | AtGA20ox1 | pEL1.2 | 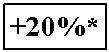 |
| F131 | AtGA20ox1 | pEA2 | 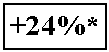 |

Significant differences (p < 0.01) compared to wild type marked with an asterisk * and shown in bold and in boxes.

The growth of the transgenic hybrid aspen in field trial was similar to those seen in the green house, which support the unexpected enhanced (increased) growth with the selected promoters, the most unexpected results were that the pEC1 promoter in construct F134 showed the same high increased growth as with the p35S promoter in front of the GA 20-oxidase gene. So here it was seen that the weak and specific over-expression with the pEC1 promoter gave the same high increase in growth as with the constitutive and strong promoter 35S.

### Example 7b Hybrid Aspen Field Trial Experiments after One Growth Season

After one growth season in the field a new set of plants were measured and the results are summarized in Table 10. Statistical analysis was according to the Dunnett's method as discussed above.

TABLE 10
Significant differences (p < 0.02) compared
to wild type marked with an asterisk *.

| Construct | Gene | Promoter | Height |
|---|---|---|---|
| F127 | AtGA20ox1 | p35S | +84% * |
| F138 | PttGA20ox1 | p35S | +89% * |
| F129 | AtGA20ox1 | pECO2 | +24% * |
| F134 | AtGA20ox1 | pEC1 | +66% * |
| F136 | AtGA20ox1 | pEL1.2 | +15% |
| F131 | AtGA20ox1 | pEA2 | +5% |

It can be concluded that all poplar plants with the GA20 constructs had a significant increased growth after one growth season compared to wild-type. A clear and unexpected effect is that the promoter pEC1 in combination with the trait gene, GA20, works nearly as good as the 35S promoter. A good growth increase with the field tested promoters is both unexpected and wanted, since these promoters might be used instead of the non-specifically expressed 35S promoter, which is expressed in the whole plant.

### Example 8: Construction of Novel Promoter-Gene Combinations for Expression in Eucalyptus

### Construct E13

The strong constitutive promoter p35S was combined with the Eucalyptus grandis x urophylla GA20ox1 gene, EucGA20ox1 (Seq ID No: 19) in the pSTT0111 vector to create the recombinant DNA construct E13, p35S-EucGA20ox1. The construct is used to produce transgenic Eucalyptus trees.

### Construct E14

The weak constitutive promoter pECO2 was combined with the Eucalyptus grandis x urophylla GA20ox1 gene, EucGA20ox1, (Seq ID No: 19) in the pSTT0118 vector to create the recombinant DNA construct E14, pECO2-EucGA20ox1. The construct is used to produce transgenic Eucalyptus trees.

### Construct E15

The cambium specific promoter pEC1 was combined with the Eucalyptus grandis x urophylla GA20ox1 gene, EucGA20ox1, (Seq ID No: 19) in the pSTT0117 vector to create the recombinant DNA construct E15, pEC1-EucGA20ox1. The construct is used to produce transgenic Eucalyptus trees.

### Construct E16

The leaf specific promoter pEL1.2 was combined with the Eucalyptus grandis x urophylla GA20ox1 gene, EucGA20ox1, (Seq ID No: 19) in the pSTT0115 vector to create the recombinant DNA construct E16, pEC1-EucGA20ox1. The construct is used to produce transgenic Eucalyptus trees.

### Example 9 Eucalyptus Transformation

A new transformation vector is constructed for expression of a trait gene in Eucalyptus. The vector backbone is based on the established plasmid-PZP (pPZP) vector system, a small, versatile pPZP family of Agrobacterium binary vectors for plant transformation, Hajdukiewicz et al. 1994, Plant Mol. Biol. 25 (6), 989-994. The T-DNA cassette is designed to contain the desired genetic elements, a selectable marker cassette and a trait gene expression cassette. The genetic elements are separated by linker sequences containing unique restriction sites to facilitate cloning. The selectable marker is kanamycin for both bacterial selection (plasmid selection) and selection of transgenic plants during the transformation process. The method of transformation of Eucalyptus may be Agrobacterium mediated transformation using a standard protocol and kanamycin selection essentially as described by Tournier et al. Transgenic Research, 2003, Volume 12, Issue 4, pp 403-411, or by Ho et al., Plant Cell Reports, 1998, Volume 17, Issue 9, pp 675-680.

### Example 10 Regeneration and Growth of Eucalyptus Plants

The transformed tissue generated in Example 9 is further treated under conditions for plant formation and root formation to get a transgenic Eucalyptus plant. The regeneration may be essentially according to the protocol presented by Tournier et al. Transgenic Research, 2003, Volume 12, Issue 4, pp 403-411, or by Ho et al., Plant Cell Reports, 1998, Volume 17, Issue 9, pp 675-680.

1. A genetically modified plant comprising a heterologous nucleic acid construct comprising a promoter sequence operably linked to a coding sequence encoding a gibberellin 20-oxidase gene product, characterized in that the promoter is preferentially or specifically expressed in meristematic tissue of said plant.

2. The genetically modified plant according to claim 1, wherein the promoter is preferentially or specifically expressed in at least one of cambium, vascular meristematic tissue, and shoot meristem tissue of said plant.

3. The genetically modified plant according to claim 1, wherein the promoter is not significantly expressed in at least one of mature xylem, stem phloem, whole leaves, whole roots and bark of said plant.

4. The genetically modified plant according to claim 1, wherein the promoter is selected from the group consisting of pEC1 (SEQ ID NO: 7, 26, or 31), pAIL 1 (SEQ ID NO: 10 or 29), pEA2 (SEQ ID NO: 4 or 23), and promoters that have the same, or essentially the same, capability of initiating transcription of a coding sequence when operably linked to said coding sequence.

5. A genetically modified plant comprising a heterologous nucleic acid construct comprising a promoter sequence operably linked to a coding sequence encoding a gibberellin 20-oxidase gene product, characterized in that the promoter is selected from the group consisting of pEC1 (SEQ ID NO: 7, 26, or 31), pAIL 1 (SEQ ID NO: 10 or 29), pEA2 (SEQ ID NO: 4 or 23), pEA3 (SEQ ID NO: 5 or 24), pEL 1.1 (SEQ ID NO: 8, 27, or 32), pEL 1.2 (SEQ ID NO: 9, 28, or 33), and promoters that have the same, or essentially the same, capability of initiating transcription of a coding sequence when operably linked to said coding sequence.

6. The genetically modified plant according to claim 1, wherein the gibberellin 20-oxidase gene product is a gibberellin 20-oxidase from Arabidopsis thaliana, Eucalyptus grandis, or Populus trichocarpa.

7. The genetically modified plant according to claim 1, wherein the gibberellin 20-oxidase gene product shows gibberellin 20-oxidase activity and has an amino acid sequence at least 50%, such as 60%, 70%, 75%, 80%, 85%, 90%, 95%, 99% or 100% identical to an amino acid sequence selected from SEQ ID NOs: 14, 16 and 18.

8. The genetically modified plant according to claim 1, having a modified trait as compared to a non-modified plant of the same species, wherein the modified trait is selected from plant height, stem diameter, stem volume, wood density, stem dry weight, bark dry weight, average internode length, number of internodes, vegetative growth, biomass production, seed production, seed lipid content.

9. The genetically modified plant according to claim 8, wherein the modified trait is plant height, stem diameter, stem volume, average internode length, or wood density.

10. The genetically modified plant according to claim 8, wherein the modified trait is increased as compared to a wild-type plant of the same species.

11. The genetically modified plant according to claim 10, wherein the modified trait is increased as compared to a wild-type plant of the same species when said plants are grown under identical field conditions for a period of at least one year.

12. The genetically modified plant according to claim 1, wherein the plant is a crop plant, such as sugarcane, pumpkin, maize (corn), wheat, rice, barley, rye, rape, forage grass, beet, cassava, soybeans, potatoes and cotton; or a woody plant, such as a hardwood plant.

13. The genetically modified plant according to claim 1, which is a woody plant of the genus Eucalyptus or Populus.

14. The genetically modified plant according to claim 1, wherein the heterologous nucleic acid construct comprises the promoter pEC1, or a promoter that has the same, or essentially the same, capability of initiating transcription of a coding sequence when operably linked to said coding sequence, and the modified trait is at least one of plant height, stem volume, stem dry weight, bark dry weight, internode length, and wood density.

15. The genetically modified plant according to claim 1, wherein the heterologous nucleic acid construct comprises the promoter pEA2, or a promoter that has the same, or essentially the same, capability of initiating transcription of a coding sequence when operably linked to said coding sequence, and the modified trait is at least one of stem diameter, stem volume, stem dry weight, and wood density.

16. The genetically modified plant according to any claim 1, wherein the heterologous nucleic acid construct comprises the promoter pAIL 1, or a promoter that has the same, or essentially the same, capability of initiating transcription of a coding sequence when operably linked to said coding sequence, and the modified trait is at least one of plant height, stem diameter, and number of internodes.

17. The genetically modified plant according to claim 1, wherein the heterologous nucleic acid construct comprises the promoter pEL 1.1, or a promoter that has the same, or essentially the same, capability of initiating transcription of a coding sequence when operably linked to said coding sequence, and the modified trait is at least plant height.

18. The genetically modified plant according to claim 1, wherein the heterologous nucleic acid construct comprises the promoter pEL 1.2, or a promoter that has the same, or essentially the same, capability of initiating transcription of a coding sequence when operably linked to said coding sequence, and the modified trait is at least one of plant height, stem diameter, and stem volume.

19. The genetically modified plant according to claim 1, wherein the heterologous nucleic acid construct comprises the promoter pEA3, or a promoter that has the same, or essentially the same, capability of initiating transcription of a coding sequence when operably linked to said coding sequence, and the modified trait is at least wood density.

20. A method to make the genetically modified plant according claim 1, said method comprising the following steps;
a) providing suitable part of a plant;
b) providing a heterologous nucleic acid construct comprising a promoter sequence operably linked to a coding sequence encoding a gibberellin 20-oxidase gene product, wherein said promoter is preferentially or specifically expressed in meristematic tissue of said plant;
c) introducing the heterologous nucleic acid construct into said suitable part of the plant; and
d) regenerating a genetically modified tree from said suitable part of the plant.

21. A method to make the genetically modified plant according claim 1, said method comprising the following steps;
a) providing suitable part of a plant;
b) providing a heterologous nucleic acid construct comprising a promoter sequence operably linked to a coding sequence encoding a gibberellin 20-oxidase gene product, wherein said promoter is selected from the group consisting of pEC1 (SEQ ID NO: 7, 26, or 31), pAIL 1 (SEQ ID NO: 10 or 29), pEA2 (SEQ ID NO: 4 or 23), pEA3 (SEQ ID NO: 5 or 24), pEL 1.1 (SEQ ID NO: 8, 27, or 32), pEL 1.2 (SEQ ID NO: 9, 28, or 33), and promoters that have the same, or essentially the same, capability of initiating transcription of a coding sequence when operably linked to said coding sequence;
c) introducing the heterologous nucleic acid construct into said suitable part of the plant; and
d) regenerating a genetically modified plant from said suitable part of the plant.

22. A nucleic acid molecule having the capability to act as a promoter when operably linked to a coding sequence and introduced into a plant, wherein the nucleic acid molecule is selected from the group consisting of:
a) nucleic acid molecules comprising the regulatory elements comprised in the promoter regions pEC1 (SEQ ID NO: 7, 26, or 31), pEA2 (SEQ ID NO: 4 or 23), pEA3 (SEQ ID NO: 5 or 24), pEL 1.1 (SEQ ID NO: 8, 27, or 32), pEL 1.2 (SEQ ID NO: 9, 28, or 33), pAIL 1 (SEQ ID NO: 11 or 29);
b) nucleic acid molecules comprising the promoter region that is located between start codon and 300, 250, 200, 175, 150, or 125 nucleotides upstream of promoter regions pEC1 (SEQ ID NO: 7, 26, or 31), pEA2 (SEQ ID NO: 4 or 23), pEA3 (SEQ ID NO: 5 or 24), pEL 1.1 (SEQ ID NO: 8, 27, or 32), pEL 1.2 (SEQ ID NO: 9, 28, or 33), pAIL 1 (SEQ ID NO: 11 or 29), or nucleic acid stretches that are at least 40%, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to said part of the promoter regions;
c) nucleic acid molecules that are promoters that are orthologous to the promoter regions pEC1 (SEQ ID NO: 7, 26, or 31), pEA2 (SEQ ID NO: 4 or 23), pEA3 (SEQ ID NO: 5 or 24), pEL 1.1 (SEQ ID NO: 8, 27, or 32), pEL 1.2 (SEQ ID NO: 9, 28, or 33), pAIL 1 (SEQ ID NO: 11 or 29);
wherein said nucleic acid molecule has the same, or essentially the same, capability of initiating transcription of a coding sequence when operably linked to said coding sequence, as compared to the promoter regions pEC1 (SEQ ID NO: 7, 26, or 31), pEA2 (SEQ ID NO: 4 or 23), pEA3 (SEQ ID NO: 5 or 24), pEL 1.1 (SEQ ID NO: 8, 27, or 32), pEL 1.2 (SEQ ID NO: 9, 28, or 33), pAIL 1 (SEQ ID NO: 11 or 29).

23. The nucleic acid molecule according to claim 22, having the capability to act as a promoter with preferential expression in meristematic tissue when operably linked to a coding sequence and introduced into a plant, wherein the nucleic acid molecule is selected from the group consisting of:
a) nucleic acid molecules comprising the regulatory elements comprised in the promoter regions pEC1 (SEQ ID NO: 7, 26, or 31), pEA2 (SEQ ID NO: 4 or 23), pAIL 1 (SEQ ID NO: 11 or 29);
b) nucleic acid molecules comprising the promoter region that is located between start codon and 300, 250, 200, 175, 150, or 125 nucleotides upstream of promoter regions pEC1 (SEQ ID NO: 7, 26, or 31), pEA2 (SEQ ID NO: 4 or 23), pAIL 1 (SEQ ID NO: 11 or 29), or nucleic acid stretches that are at least 40%, 50%, 55, %, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to said part of the promoter regions;
c) nucleic acid molecules that are promoters that are orthologous to the promoter regions pEC1 (SEQ ID NO: 7, 26, or 31), pEA2 (SEQ ID NO: 4 or 23), pAIL 1 (SEQ ID NO: 11 or 29;
wherein said nucleic acid molecule has the same, or essentially the same, capability of initiating transcription of a coding sequence when operably linked to said coding sequence, as compared to the promoter regions pEC1 (SEQ ID NO: 7, 26, or 31), pEA2 (SEQ ID NO: 4 or 23), pAIL 1 (SEQ ID NO: 11 or 29).


SEQUENCE LISTING (36 sequences)

SEQ ID NO: 1 - DNA, 1084 bp, Eucalyptus grandis
cagaagcccttgcgttgactacagtctgcatttcagggcagttaagaaagagggaaaggaggagagggagagatgtagaaagatggagagtccatcagaaacagaaactgactacagccatttatttataggaagatgaggactctcgagactcgtggcgaagaacgagtcatcaagcacggggcccttcgatgcggaatttttttggaggtcctgaagatgattattcatttccccaccatgtctataaattcttcgaatttgtggcaattcaagttggggaacaaaacacggccagacccaaagaggaaaatgagtttacaacataattcaaccgacatgaaaaattagttcttgcaaatatgatgccatatagatcattcttcatagttaagctactatgtggttgttaacatgatattcgtgttgatacttataaagagaccaatcaaaatcgatccttatccttaaaaagtcaatatattttcggggaggcctattggaatactaataatcatgctgcctaattttgtcataagaatgtgagatctcgatataatcaaaggtcgtgagatcatgacagatgtagataggtaggtttcttaggagcaaagcaaaatatcggggacactgctggccagtaaagcccatgatgcagataaaggaacgaatccctccctccatctttggaagcccaacccacctcagaggccaaaagctacgttgacaggagaatgggagcagagagtcgcatcgttcaacgacgtgtggttggaccgacgaaaacttcaaaagccagggaaaaagaaagagcgcctgtgatcctttacgtaaatacgcgggggggagttctgtggtgcgtttcggttttttttcaacaccgaccctcccagttgtcttccttcctgcctaagatcggttttacttttagacgatcgattagacgatcgatcgctctctctctctctctctctctgtggcctcacgcgcatttccataggcaaccaacaacagcagcagaaagtagagttcctgcggcgatatcgcgcgatccgagtcgcgagctgaggccccgaccccgacg

SEQ ID NO: 2 - DNA, 2000 bp, Eucalyptus grandis
aaaaacacggatatgatacaaaatagagagagagagggatctattattctcacttggatgtattgtaagaaatcacaataaatctttcattcttgctagaaggggaagttgatacccccaatctaaacttgttctttttagtccatctattttggtcaattgagtagctttccctctaccaaactctgaaatgttctaaaataatttctcaaaaaaagatttttttatcaaaagaatagaccttgaatatatattaagccatgtcaaaattgcaaatcacagcaattacaattaataatcctaaaacgaaaaatcaagcttgtaacgatgattgctacaaaaaaaattatgaattgcaatttttttggggagttcactaaattatgtaaccttcaagaactgagtaaatctaaaaaaatgttccaaaactctagtagaattacgatagtaacttacacttgctaaataatctaaagaccccaaaagaaataaagttattttagtaagtgaaacgcttcggattctgatagtggaattcccagctataaaatctcaagcacatcccaaacctatagtattaaaaaaattgccaaatcttcgaacttggagaacttttttttctagaaaccgatcaaatttaattttagtaggcacatgcagacatgtgccatttcaccagtaaactaaaaaccaagaaaagattaacacggaatgacaacattctataataaatttgagaaaatcgcagaaaatgtgcaacaaaatcgagagatccaatccattcactagaaaaatgtaatatcgagcgcaattaccaaaaacatcgagttcccatacgcgcgatcacacaagacatatagcaaaaataaagaaaagagattatttccaaaaaaaataaattggatttatacacggcatctagaaagaggccgactcttggatacattaccaaagaagaaattcactcgcaagaccatccatattctgtagaagattctagaaccaggagctcgcgaaaggtagttgccaacttgctcatgtcttgtctgtccgccatcaacctttcatcggcttttgacttttcctcctgaccaaaaaaaccctaattcaattcaaaattctcctccggctgctgggtttaggtactcctaaattcggcgggcaatgccttaagggccacaagtttttcttcagtctcgcacaatttttttttttttttaacaacacataaaataatagtaatttttgcgggcgtctaatataataaagaatagatcaaaattgcttgatcaaaatttactttgcgctggcgcaatatgctcagtcttttgttcttgaattaatcacttcttcgatatgactgtcgctaaaagcgacatacgcgaaacaaattttggtacgtaagatcgatacaaattgttcacaatttcttagaatttctaatatttcttttattttttgcaaagggggaggcaaaaaattaaatgagaaggaaataatgttagacaagcggaaccattgaaaacctcctcctacccaagcccaccggctctcagatatgctccgaatattcccgcttctcgttttccccacgctcgcttcacgcgcgtggctcacaatcctaccctccgcgccggcctccctttcgaattccccgagctcggcgcgtgcccgtgatgggcacgacattcccgaaccagcgggctcccaacagagtcccgctttcaactaaatcagcggctcgaagcatcgttcttctcgctatcatctttagggatttcgtttcgcccaaggcagagagaaaaaagagagaggctgactcgtcgactcacgtgacccggcccgagcggactcaccgttctgactcgcttatttaaagcgtcttactgtccctcaccctgttgtctcaaaccactctcaactcactctcatctctacactgctttttctcgtcttctctcgctagccatttccagaaccctcgtcgcgcgtcgcttgcc

SEQ ID NO: 3 - DNA, 2000 bp, Eucalyptus grandis
aaatggatatgactacacaattattttcgagtgcatactttgtccatcaaacaaaaatgcctgaaaatttctttattaatgggctcgacaaggatgctaattgctagtgaaattcggtgatttgctgctgctgctagagacatgttagactcttgtccatgttcgtgcctcttgggcaattttgttgtctaattcgagagagatcacaaaaaaacatggaagtgtgaatctcttctcgtctacttgatatgtagccaaatcacatgaccttaagagcctatctttttcatcaattcgccaatcatacgagggaggttagatgctgattcgcgagccgtaaaaagcccattcatcgtctaactagccatgcctaggtgaatgcaaccgcacaattagctccaaaagcatatatcgttcgtcgaacggaaatgtctaagaatttatcgacactacaaaaaggatcctgattggatgtgttggactcgtgtctatgcgtgtcccctttggacaacttggttgtctaattcgagagaaaatacaagaagccatgaaagtgcagatctcttgcttttccttttagagtacttgatatatggccaaatcacatgatcctagaggccatatctttcatcgtttcgccaattatatgaaggaggttaggcactaattcacaagctgtaaaagcaccagcatgttaaacaagtaaacgaaaaacgtcatacattcaaaaatgtatacacataacgagtatcttggaatagaatactatgttattatccagtttcgatacgacggagaatcgccaagtcatgatcttctcgcgattccattgatcggaacaaggttcgcctatgctccgacagtaaatcctagggagggccacccccacatttctaaatgctaacaagaatcaagaacgagctcgagacaaaaaagatgtcctgtaataaccttagaagtcctgcattgattcgcacacgtcaggcaatttttcattttggatggtaaccaatccatcgaagcttggctcaatgcatacagaatagaaacaagtatggggggcatcgagtattaaatttggaaatgatgagagatgggagaggccatggtgcgagaacgcagttaattcttgctctgcaacacacagtgaaagagagagaaataattcgtccttttcgacccctgaaatggatcgccatttacattttacccccatctctgcaagaaaacagctcccccccagcaagaaccgagaagcgaaagcgacccattattcattacggcgtatgtaaccaagcatcccaacattcaagaaagagagagagaaacggtagtagtggggtggggaggagggggggaaatgtagagagagaaatttcaccacccagagtgtcatagcgcgcagagagagagagagtatagcgttgcaggcgttgttgcagaaccgtggcagatggtcttttaaccttttctctcgaaataattcgctccctctctctctctacccacgtctatctctctcctcattacggttctattctggggtggctctttgcctgggagcagtgagcattatatgctcatcgaaggaggagaaaaaaatgagaacggaaaagcaaaatgcagtgtaccccatctttctctgcaggcagagagagccctgtgtgtgccagagcgatgtgatggaacgagtaaaagtaagctaatctcagacttgaggctgtcataacatctcagagggagaagaagaagaagaagaaagcccaagaatttacttcttggttttttcaaaatgactatcaaagacccaacgaacatgcgcttctctttcttcagctcctccttcctatctctgcaggcatcgtgtcggtgagctcggagtgggctccgaccgggcaagctttgcttccgaagcattcgttcgtaacagagtccaaagttggctcctttgccgcgaattctggagtttgaaaagtgtgtggttggctgggggaacggcc

SEQ ID NO: 4 - DNA, 2500 bp, Eucalyptus grandis
ctcacaatccatatgcatcgcgggccattatatcttaaccgaattttagtgcgccctaaaatgaaatcatgcagatattcattcgttaaatcgcacgtcttttcgcaagagcacgtcataaataatgctctagcggatgcaaaagcgtgtgctgttaatgtcgaatcaaaagctgtaaaccacgttcgaggggctaaggcaatgtttttcgcaggaagtcgcagacaggagaagaagagatctaaaattagatactcaagctgtaaatagggtagagagagggggagggagtactgtacgtaggaagtgactggagggtgcacagtgtccctcccggtcatatctatcttaggccctgcgcctctgaaaaaaaagctacccagttttggttatcttctttgctgtcgccttgggaaaactcgttattttattattttacaaaaaaaataaagtatttttttggtacgtaccatctgtttgatatatactgtgctggatttttttttttttctgggtgtggcggtcgatactttggtggatatcgtatgatttctgttttttttttttgtttattcattttctggtccattttcttttgagttgcttttattataacagaactagtgataatgatttctctgatttcattcctgaaactacattgtaaaatcatgtagaacttattagcagacaacattaacatttaaatctggaaaaaaaaatcataacttgtttcttaaaattagaaacaagctggaaggggccatacccgcgtggattatctctccggacattatcataagagttgtatgcccgccaacattgttcattttgaactagccaattgttattgcctcatcataatgttagcaaaaatggaatcggagttatcctgaatgacaacatgatcaaacctacactttggatacacattcctgaaattctaccctctcaaacttgtcacaacataaccatttaaaggagatgctccaaaagcttaattcctcctattttaaaaaaaaagatcgtgctttacatgagattcatcttgaaaatttatgtacaataaacaaaaagaccacccaaaaaagttagaatagaagattcatccacaacgagaagtatgacaagtgtacgagtcattgattttgcttaggttgggtaagaatcgggaattgaatttcttggcaaaacaaagattctcaatgcaattcaaacaatgatcgcaaaagtctgtacgtatatatagagtttgtaaacgtcgatgatttcaatattaaacaaatctcaaatggcaaaaagatgaaagtcgctctaaattagccctatgtgagcaatatttgtggatgatcatttgccccgtgacattaatatgtggcctttttcctactagacgctcaaaatctaataaatatagacttttgaggccacatcaatagtcttcataatttactaacataacagtttttgggctaccgtgagaataataatttgatgcgagtcacaaattattacgaggtacaaattatcgaaagaatggacctcataataatctacaattatttgttatttagttttttatgatctaaaacaaactattattaaagaacatcatccaattattactcattttcttttttattggaaagagtccataatttcccaaataattataatgtaatgtttactgtgacggaaaaatcaaatgctacttattttttggttggctgagagctggaccagaaagaggcagacgctgtaccaatatatgaatgtcattaatgtaaaatagtgcgttgggattctatggagggagggaaggggaggatgtttgtagtttccattaaataaatatagtgtggaactttaatggtatcaggacgacaacgagccaaagaatgtccggaggtcgctgtaggagcacgttttctgggcttatataatgcgcgcgtccgggatttgtgtgcttgtgttaagtgtcgagttttgttggctgttgtctcattaagccaaaaggccaatccataagagaaaggaggaaaaaaaaaggagaaaagcagagagagagagagagagagagagagggagtagggggatttctcgcacattgccttacacagatccccatggcttttttatgcagggatactacccagcattcgcttggagctgagctctttttattctctttgttttagattgaagtgaagggaccatttctctctctctctccccccctatatatgagagcttgagaggctgtgggtgtgaagttgttgggttatattctcggaactagacaacaacaacagggaaaaaaaaaaaaaaaaacccaagtgtgtgtgggtgtgtgttttctctctcagatgcaagaagatttcgaaatgattacttcccttcttgccaatgattgaagcctcgagctcgtgcttgcctcgcaccacaatgagacatgcttaacgaagtaccgactttttaactgaacgaggaggaggagaag

SEQ ID NO: 5 - DNA, 2700 bp, Eucalyptus grandis
aatgcaaaatggccgaaaaagagaggagagaaaagaaaaaactcagtcccgtctctcctccgcacgatccccatccccatccgacgcgcctgatctctccccagcgagatcagaagacgctttttacaccgaaacggatcgttctgatacgggggacggggggcaggaccgagcccaagtccccggcacggcaaaagcgaatcgcgagcaaagctcggtgcgtactgcagagaattcggggcacggagcggtgtaaaatatgcaggggagaaatcgcgaaataataaccggaagcaggggagcttacgatatcaaattccctcgtacacgtcagccgccgtggggaccacctccttcttttgtcctcttgctcctgctgccctcgcatgctcctgctctctactctctctctctctctatactctccatccgtgctttgctctctcttgtcacttcctgtcaaagttgcttgctttctctctctagcttagcttggagcttacctactgtactctgcttttcccccccctctctctctctctctcgctctgaacgtttttcagtcatcgagccgagctgaactcctctctctctctctctctgggtgacccttcttcccttttctctgcgcttccgtcgaagtgaaagagggcgccgaggttgtcgtccgagatgcgtcggagaaggacctctctcctgatttcatgagaccgaccagtgagtaagctcgctccgatgactggagcttcctctgacctccctttctttctttttcgggtgctgagcttgtccagcgtcttggccgtcgtcaattttacgctttcgtgtgagctttgcttgctgtctttgtgggtgccgtgcgggactcgattcgtctggtaatcgcgaatgcaggatcgatgctgacttttgagctcccgaaggacggacccttttatctttactagcagaagaccttaaaaagtggaggttctgatttctttttcatatttttctttggctcacggtctcaaatgagtgatcggtttcggttcttggaaagatggcatttctttttcttgaaagaacagcatctggtgaatattttattgttatctcatgcctagaaaggttccttttatttttattttctttttggttctgggaaagggttccagattccattatatctcatcactcggttgcatattcaattgggctctttggtttccctcgggttgcatctgtcttgtaccctgaagaatttggcatccgccgttcttaatgcagattcgttcactttttggagcttctgtttggtctctctttctgcatgacattcttgcattggttggtccccgtggactactcacaattggaaggttcagatgcttttctgggtgcataatttctatcagatgtttgtagagatggactcctttggggggtcatcctcctacatctactttgggggctctagtatgctgtattctgtatggttttaagctttactggaattttaacatcctttcctcttgtggtgtttggttgatgagatgcggatgatagaattccgaactcatcatttagactttcatttgtcgatttaaagactgataatcagaaaggagaattcaacgcaatttcacgttcaattaacttcttgctccacttaaattgcgggtgcagttcaggggccggtttggattctctccttcaatagatttagatgggtaatctctttataatgttttggagattctggaacaagcatctaggagtcaatgcacaaattgcaacactcagcagcatcaatcatagtgttcaccatttgtttggaactgtagatttgacgatagattattgatagggggttttctggattttctgtatagttagatttgcttggaagggacttgtggatgatctagggcggaataaattggtattagatttcaatctctttttgtctgagctcaaattccttgtttcaatgcattttaactatagttatggtactagatttctttctttccttggctgaatgtatgtgaatattgcttccttcatgaaaattgttgaaccagagaaatgcatggaaacatagtgctgcttgcttcgggaattgaacaatatagctggaccagtttggtcgtgtagtgactggaagtttgcctagggtaatatcattttgcttacaaaaccttaacaaaagaatgtgggactcttcattctgttcttggtgattgttctttcagacatagatttcgcttttcagttgttatatttcttggattaggtataattagagaatttgaacatcagcctgctcatgtcctttccttgaaaatgacatatccacccagataccctatgaatttatggaggataagtgacgacctcggctgtaaatgttgttacacaactgttttctctttatgatgctagggagagattgccgcttgttttttcttgcttgcctcactaattggccattgtcttttatagtttcccctaggtctgctttcatttccttggataaacctgtctcctccttgggattcttttcttttctcagaccagttaactcagctaaaatgttgtgtattatattgtgatgtgagaaattacttctaatttgtattatcaccatcttcttctgtagccacactatgaaagatctaagtttgaggcaatgggaagtggtgtctacacctggca

SEQ ID NO: 6 - DNA, 2500 bp, Eucalyptus grandis
ataaaaaactcctaagcataggtgcttcgagcatcctaaatgcgagttctcttattgcaagagattgaaaactctcaagcatgagaaattgagattcccaaacaagattcaatgaaaactctcataaaagtaagagcaatcgaagaaagccaaccataattgtatagtttttacctaaattttaggttgaagaagagcgcaatttgagtcgatgtggaaaattgatctccaaaagatgtcttcttggatttggaatatgagataaaactggacatgccacattactaagtgcaactttagatggagtgataacggaatgagaatcaagtaaaaattagtggtgttttatgagacaaaaaaagtttaggttagaataaggacttgacataaagtttaaggtaggattgtgattagaccaaaagttgagcatttttaatgaaaaaaagaaaagattagggtagaattggaacttaactgaaatttgaggaatttttagggtattagggtcacataaaacacatgataattatatcctagaaaaagtacttcaatttttagcttgcatatcatcttttatttagccaattagtgatgtaataagtaaattatcactcgtatgccaaaaaatatcaattataatatatcatcttcacagagaaaatgtaataatgattttatttctctttaataggatcagattagtaataaactcagtcattaaatgcggcatgtatataaaattttgtagttatagtggtcctataaagatatgcaattttttttttctcatttataacgcagttgatttgataaactcgtgataatatgaagcttagattatatattagtaatgtctaatttctgttgcgtattttttcatccctcatcccacatcataaaatttcagtcttctatgttaccaacaaagggtgtaaaagaactcatcgacatcgagttaaaaggttgtttactaacgtttgcatagcaatttctttttatttttgaaagcaaacaaaaacaattattgaaaaacattgtttgggtggttacaaaaaaaaattaattatgtaaaaataaaagaaaaaaaatgacacaaatggttactcttgccaagcctcgatcttttgaccttaaacaatgcaaagcctttactactgggctactatgttaggtggtgcattttagatagaaggatatcacaagatatttgttattccatgtccttcagacagcaattcacacttactgatttgagattttttgtatgcacaacgttcttatgttctgtgaccatatttactattgtcatagtgttgtaactagattatacaattcacttggttttcttcttttatgtcgagcgtgctaatacacaagttcccttacttgattaaaagagcatacataagatcttgaagactgcggagacataaagattttaactttgagggtggttaaagagattacatggaatattttaaatttaaataactatcgatactcacatggtaggttaggaaagacaaataactttctaaataagatagtggcaagcgattttattcaacaagtatcaaatgcaaagtcctcaccgaatctggggtttataatagttgagtcatcttggtttgtccatataaggattctagtcctttacctaataggttttttcaaggtttatgtgatgtgatgggcttgcccatcttgttgaaaacatcaagatttttgctagtaagcgagacagactttacaatagtctctatataactggatcgattaagacccgactaaactaaatgatatataaacgatatatcggaaacctaaagtccaactcaacaattgacgtagatatcaacgtatgatctactccattaatctgatcgtctctagacaagtgactgggtagtatgtgaaaagagatattatccaatcgatgcactttggattaagggttttaaacttacacattgattaaaatattaagaaaatttcgtgaatttgatttataatatgatacgatagactctttattaaatactaacatgacacatttactctccactcatgatttatatcatgctattttgtaagtcaattatataaaatctagagaggccatgcctcagctcttgcctttggtctaacccagccttgaccccacaaaaaaaaaagagcaaaaatctttccgaggaaaaattgcacaaattctacattaaacttttttgcagatatgaacaaatattttttttttctcgaaaatcttccaagacaaaaggcgaggccccttcatctccttcctcacgcaatcgcttggagcgccctctctccctcactccctccctcctctctctctctctctctctctctctctctctccatcccacattcttgaaagagacacatcagaagcgcaggcatgaatctgggcaccggctcgttctctttcctgctccatctataatgagcaaagccttcgctctcttctccgcagaagggc

SEQ ID NO: 7 - DNA, 2101 bp, Eucalyptus grandis
caacctcgtggtaatgcaacgaaactagcccaaatatatgttgatagccaatttggagtaactctaattttgtttgcgttggtataagatgcaacaaggtacataaaatccaattccattgaccctgaattgtgtgaggtcttctcatgctggaaagcactgaaaattacaaattcacccccacccagagtcaatacgtagacatacatatgattttacgcaatcattgaagatcaaagtggaataaaataatatttggagggtgatagtgccactgccatggtggtgcggtgctttaaccctggagctctctacacaattctctctctctctctctttcggattatgaaaatgaaagtggggttacgtattgctggttttgaactgtttgggaaattgaagcaagctgttcccaaagaaaaaagcacgccatttattggcaaaataatattaaacatatatctggggcattcagatagagttttctcaacactttgcctattttaaatatttattattttccaattgtatattccacttccattgcctaaagacgacggcattcagttttccccccttctttattcatttatttccccccccccccccttttcccgtcattggactgcgtagaaaagaggcaatttcgagtatagggattggagggatctttctttcgtttttttctttttttttgcttgaattatttttactattttttttaattatttgcttcattccctcgcacctcttcaactttatggaagaaaaaggttgatagaggccaccgaaaagcatggacaccttatcatgaaagcgagaaaaacacgacccgatttgacgggccaacacacacccacaacacaaaataccaaaaaccactcgagcgcctcgcaaaaaattcacacgcttgttccatcatcacattcacatctccatctccaccttctattccctcttggctattctctccaccccctttttcctcggtctctgttgacgagcatgatggagaaatacagagagttttgtcatgtttttgaagaagagattggctccttctgctggcttgcaatgaagaagtggagagctgctgaatggctccatatccttccaatcttccaaaattccacttgattaggatggacaatagtctgtcaaaaagggaaaataaacttatcctgggaaaatcatcgtttcaatcttggccctttaaagttgaatatcaagattattcgcgttatttacaatttggagaagtaaactgagtcaatttgctccaactcgacatgattgtagtccataaacctttaaaggggaaaaatgacatattgcaattgttttgctggtgagaaatcatgatataaagaggataaaattgagttaggcaattgagagcataccaatccatgcaatcaaaagcactttattgaaaaaaaatcaaataaagatatgataataattcatttttctcaataatggaaaaataccttatcaatctcatgaatgagataaacatgagttaaggatctaggtggatgcatctatattaaactctttacggtaggtggtgtagaaaactatgatcccttttatgagcctaatccacacacaaatatgttttttaatcactagccaaactctacaaattaaataccataagaaaaattaaacaataagaaaggcataatttaaagtattataaatttccatgctagtgcatttaatgcaactgaaagagcacctcaaagcaaaatggcaatcataaacagtttctcctttttcctttttgctccagtgcaactttagctttttcatcgtgtgggtaatcaatctccaagtcaacctcgcctcatcatttggtcttgggttcttctgaacaacccaccatgcacctcatttcccactttcccactttcgctctctctctcgctctctctctctctttctctctctccctccctctctccctccctctttttcgttttgtttttgtttttaccttttcaacaggagtgtgtcattccctatataattgcctctctcttttagccggatcttcccctcatgggaagcagaagcatctcgtttcaggcaaagc

SEQ ID NO: 8 - DNA, 600 bp, Eucalyptus grandis
cgacgcaggtcagacgactgagtactgaaagcattggcctatccgtcctcattgaaccaaacaatcaactgatgggtcgggccgctccgagattaagactatttgtcagagaatttttcccaattggtggtccattttcgtcctttaccttattcaaatggaagtctcgagatattcatgtaaaatcaacgaaaaataattttctaaaatgaaaagaaacaagaggaaaatttcgtgtgacatataagcagacgggccccctgtcccctgtcgcactcaaaacacaagatgacaaactcttctcgattagcacccattctttagaggcggttttgtgtccaccgcgtctcttatccggataaaatggctcgagtgcttaatccgtctaagcccacaatttttttttcccattttctcatttaatttacaatttttttccttttttccctaattcgagtggaatttacattttagaatttagaggtcgagtggagctataaatagaggggcacggtttgcaatgtaccctcaagcaactaggagaaagtgataagccttgtgaggtgttgaagacagctttgccccaaggggagatcagtg

SEQ ID NO: 9 - DNA, 1800 bp, Eucalyptus grandis
atttgtcagaaagactaaattggtaaatcttcatagggtttaagactaaattggtcaaattgaaaagtttaaaactgaatcagtacccgtataataggcttaattattttttgataattatcttgtcatatcaatcccctaatatcttgaggcaattaaggctttactcacaagatcaaagacaactgaggctttctgaataggaaaattgttcaaaaaaccataaatttattgtgcaatggtcaatttaattctaaatatttcaattttgtaatttagttttaaaccttttgacaacatgataattcaatcataaactttttgattgtgccaatttaattataaacttttaacggattgtcaatttagtcattttggctaattttggctagatatcgttgatatgatggcctagtcaacgctaactgtcctatgtaacatagtagacgtttatgcatacattaatttttaaaaaataaatttaatttttctattttttttttatattttctttttccactttttcaaagggctaatggagttactagccattgaggaagggcagtgaagcccttgctagtggtaggtgagggccacaacctcgccattccttgttgtggtggcgatagcctaggcaagggctcatggcacccttgccagccatagtgagggcttacgagcccttgccatacctagccagccatgacaccctcgctagattcagcgagggccgtgaccttgtggtcgagaaggcttcgcaatccttgcctagggcccaacgagggtcgccgattcttggctagtgaccctttgtaaaaagaggggaaaaaagaaaaaagattactaaaaactttgaataattttttaaacattaaaaattgttcacgtcagtaacgtccaatcaaaattaattggaatgaccaaatagacaatccgttaaaggatttaggacttatttggcaaattattaaaattttaggataaaattggcataattaaatgggttaggattggattagtgaattatttaaaaatttaagattaaattgataaattttaaaaggtttagaattaaattgactgtcatataataaattaataattttttggatatcttttccctttcagatccataaagcgtggtcacaaattttgaactttttgcaatcgatgtcgatccaactcggatccgtttaaaaccgacgcaggtcagacgactgagtactgaaagcattggcctatccgtcctcattgaaccaaacaatcaactgatgggtcgggccgctccgagattaagactatttgtcagagaatttttcccaattggtggtccattttcgtcctttaccttattcaaatggaagtctcgagatattcatgtaaaatcaacgaaaaataattttctaaaatgaaaagaaacaagaggaaaatttcgtgtgacatataagcagacgggccccctgtcccctgtcgcactcaaaacacaagatgacaaactcttctcgattagcacccattctttagaggcggttttgtgtccaccgcgtctcttatccggataaaatggctcgagtgcttaatccgtctaagcccacaatttttttttcccattttctcatttaatttacaatttttttccttttttccctaattcgagtggaatttacattttagaatttagaggtcgagtggagctataaatagaggggcacggtttgcaatgtaccctcaagcaactaggagaaagtgataagccttgtgaggtgttgaagacagctttgccccaaggggagatcagtg

SEQ ID NO: 10 - DNA, 2683 bp, Populus trichocarpa
ggggaatgataggctgacaagtataaaacatgcttggtaactaaaggtaacaagcaagagtacgatgtcgattactaagaagtttttgttttagttgccgggtatgatacaataatattttttctttggtaactcaaaatccatggtttatgttgaatgtaaaaccaatatttttacatgaagaccttggttataagagagttcttcatcattaaactgattgttcaagaacatcgatataaaggagaaatagttagatattagttgaaatcctattccaagtagaaattcttgatagaaaaggatttgtgtctaaaatccttattgaattagttaattataaatcttattggattatgattgttagaggttgtctcctataaatataaaacttttaccagattttctgtccttctgtagtggttgaaaaatgttgcagggccatgatgcacgctatttttgaaagaaaaatatacaggagaaatagacatgttgaaaacaaagaaaattcgaggcacacgcacttggtttttatagttttgtactgggcacatgggctaaaatctttcaggcctaaccatgcttggttttatttatttatttatttatttggctctaatttgtttttacattttacaataaaatcaactgtaaataaatttatccagacattctcttttctagaaaaaataacgtgtcttaatactcgaatatcgcttcttaaaactcacatgtacatccaatttatttttattatatgagaaaaaaatagtattatgttccaagcttacacttgaaaaaaaacaacgaaaaggataccgtttgattacgaacgcagcatcggaaaataaaataagaatttcgactttatcctcccggaggggcctttgatcatcattcccggaatcttttaaatatatcacagacccacttccccaccttttgccgtggaagcaggtgctcactccacaccgaatgacccaaagtcgttaaaaaagcaaatggagtctctgttctttttcgcatttatattcaaatccgaaaagaaaaaggtttaattttacaccacgcgctctatatagcgtggcacgtgacaatcgcgttttgttttttttacatcatcttaagtgattttcttttccttttcaatgaaaatatttaccctttcttcatttgtaatttcaagagattatatattgcctgcactttaacatgacgtcaatgaaaccgttaacccctttcaaaataaattccgacatctgtttttttatccaaagcattctgtatgtatcttttaaatttttgaaaaaaatggttttttataccaacagctcccttttttttaatattttatttattctggtcggaactaatttctctgattgaacttcccttttaacttttttttttgtaattataaattaactttattaagctaaaaaacaaatattttagaggtaattatgagtacgatagcgttgttatttttaaagtatttttacttataaattcatttaaataatattttttattttttaaaatttatttttgatattaacatatcaaaaataatttataaataaaaaaattaattttaaataaaaaaattaaaattttatcgaaactcacaccagcaccgaacgggccgaagttgctcacttcacctaatgttttatagataaacaaaaagtcacgctagcaattcacagggaacagtcaccaccaataccatgctgccagatgaataaagcagaaagtcaaaaatggtcacatcgatgacccgcataagaagctttcaatccaaatgaacggtgggtgatggggctgtgagagcaatgcatggtcacagcaggaaggtgacccgatggggcctgcgcctatgtcccaccggtaaagtgtatgtttttgaatcgaaagctgtgaaccaaattggagcagtatttaagttgaaaggaaaaacaacagatataagttggattgcagagtggcagtgtactgtatgaagtgactgcagggggcagatgtcatgccgatcaaatctatctaaggcccttcgcacagtaaaataaagctaaacccatttggttactgaccagcctcaccctattggctactactacacacccactagatttaactgtcattgagagagagataataaagttttatgatagtatcttctgggtgtttttctggctaatgaggaaagtatatactcattttgttagctgttgaaacttgaaaaaggtttcggtggtatattggatctggcttcttttatttcctagggttttaagggggcgctcttctgaagggaaaaaaaaaaagaggttagtagagagagagagggagttgggagggagttgagagagataaaaagctaaagacgtggtctttctcatatacacagatccctatggaatgcaggggtgctacccagcattgcttgttagtattcattccttttgtttagtttaaagagaccaattttgttctcatttagagagagagaaagagaggagaattttgtgctagtgggtgagtgcatagagcgaagagttttgggtcagaattttttttttcttgcaatgagttctggctgctaaacaacaacaacaacaaaaaaaggaaaaaagaaaaagaaaaaaccaagctttcatatggaagtaggcaagattttggg

SEQ ID NO: 11 - DNA, 942 bp, Cauliflower Mosaic Virus
gatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 12 - protein, 377 aa, Arabidopsis thaliana
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCIKPDLTLGTGPHCDPTSLTILHQDHVNGLQVFVENQWRSIRPNPKAFVVNIGDTFMALSNDRYKSCLHRAVVNSESERKSLAFFLCPKKDRVVTPPRELLDSITSRRYPDFTWSMFLEFTQKHYRADMNTLQAFSDWLTKPI

SEQ ID NO: 13 - DNA, 1134 bp, Arabidopsis thaliana
atggccgtaagtttcgtaacaacatctcctgaggaagaagacaaaccgaagctaggccttggaaatattcaaactccgttaatcttcaacccttcaatgcttaaccttcaagccaatatcccaaaccaattcatctggcctgacgacgaaaaaccttccatcaacgttctcgagcttgatgttcctctcatcgaccttcaaaaccttctctctgatccatcctccactttagatgcttcgagactgatctctgaggcctgtaagaagcacggtttcttcctcgtggtcaatcacggcatcagcgaggagcttatttcagacgctcatgaatacacgagccgcttctttgatatgcctctctccgaaaaacagagggttcttagaaaatccggtgagagtgttggctacgcaagcagtttcaccggacgcttctccaccaagcttccatggaaggagaccctttctttccggttttgcgacgacatgagccgctcaaaatccgttcaagattacttctgcgatgcgttgggacatgggtttcagccatttgggaaggtgtatcaagagtattgtgaagcaatgagttctctatcactgaagatcatggagcttctggggctaagtttaggcgtaaaacgggactactttagagagtttttcgaagaaaacgattcaataatgagactgaattactaccctccatgtataaaaccagatctcacactaggaacaggacctcattgtgatccaacatctcttaccatccttcaccaagaccatgttaatggccttcaagtctttgtggaaaatcaatggcgctccattcgtcccaaccccaaggcctttgtggtcaatatcggcgatactttcatggctctatcgaacgatagatacaagagctgcttgcaccgggcggtggtgaacagcgagagcgagaggaaatcacttgcattcttcttgtgtccgaaaaaagacagagtagtgacgccaccgagagagcttttggacagcatcacatcaagaagataccctgacttcacatggtctatgttccttgagttcactcagaaacattatagagcagacatgaacactctccaagccttttcagattggctcaccaaacccatctaa

SEQ ID NO: 14 - protein, 380 aa, Arabidopsis thaliana
MATECIATVPQIFSENKTKEDSSIFDAKLLNQHSHHIPQQFVWPDHEKPSTDVQPLQVPLIDLAGFLSGDSCLASEATRLVSKAATKHGFFLITNHGVDESLLSRAYLHMDSFFKAPACEKQKAQRKWGESSGYASSFVGRFSSKLPWKETLSFKFSPEEKIHSQTVKDFVSKKMGDGYEDFGKVYQEYAEAMNTLSLKIMELLGMSLGVERRYFKEFFEDSDSIFRLNYYPQCKQPELALGTGPHCDPTSLTILHQDQVGGLQVFVDNKWQSIPPNPHAFVVNIGDTFMALTNGRYKSCLHRAVVNSERERKTFAFFLCPKGEKVVKPPEELVNGVKSGERKYPDFTWSMFLEFTQKHYRADMNTLDEFSIWLKNRRSF

SEQ ID NO: 15 - DNA, 1143 bp, Arabidopsis thaliana
atggcaacggaatgcattgcaacggtccctcaaatattcagtgaaaacaaaaccaaagaggattcttcgatcttcgatgcaaagctccttaatcagcactcgcaccacatacctcaacagttcgtatggcccgaccacgagaaaccttctacggatgttcaacctctccaagtcccactcatagacctagccggtttcctctccggcgactcgtgcttggcatcggaggctactagactcgtctcaaaggctgcaacgaaacatggcttcttcctaatcactaaccatggtgtcgatgagagcctcttgtctcgtgcctatctgcatatggactctttctttaaggccccggcttgtgagaagcagaaggctcagaggaagtggggtgagagctccggttacgctagtagtttcgtcgggagattctcctcaaagctcccgtggaaggagactctgtcgtttaagttctctcccgaggagaagatccattcccaaaccgttaaagactttgtttctaagaaaatgggcgatggatacgaagatttcgggaaggtttatcaagaatacgcggaggccatgaacactctctcactaaagatcatggagcttcttggaatgagtcttggggtcgagaggagatattttaaagagtttttcgaagacagcgattcaatattccggttgaattactacccgcagtgcaagcaaccggagcttgcactagggacaggaccccactgcgacccaacatctctaaccatacttcatcaagaccaagttggcggtctgcaagttttcgtggacaacaaatggcaatccattcctcctaaccctcacgctttcgtggtgaacataggcgacaccttcatggctctaacgaatggaagatacaagagttgtttgcatcgggcggtggtgaacagcgagagagaaaggaagacgtttgcattcttcctatgtccgaaaggggaaaaagtggtgaagccaccagaagaactagtaaacggagtgaagtctggtgaaagaaagtatcctgattttacgtggtctatgtttcttgagttcacacagaagcattatagggcagacatgaacactcttgacgagttctcaatttggcttaagaacagaagaagtttctaa

SEQ ID NO: 16 - protein, 385 aa, Populus tremula x tremuloides
MAIDCIKTMPSITTPHHHPKDQDQCKDDGKSFVFDAQVLRHQTNIPQQFIWPDNEKPNINAPELQVPLVDLGDFLSGNPVAAVEASRLVGEACKKHGFFLVVNHGVDKTLIAHAHNYVDTFFKLPLSEKQKAQRKIGESCGYASSFTGRFSSKLPWKETLSFRYKAEENSSKHIEEYFHNRMGEDFAEFGTVYQDYCEAMSTLSLGIMELLGMSLGVSREHFREFFNENDSIMRLNYYPPCQKPDLTLGTGPHCDPTSLTILHQDQVGGLQVFVDNEWRSINPNFDAFVVNIGDTFMALSNGIYKSCLHRAVVNSQTPRKSLAFFLCPKNDKMVTPPHELVDTCNPRIYPDFTWPMLLEFTQKHYRADMKTLEVFTNWLHQRSFS

SEQ ID NO: 17 - DNA, 1158 bp, Populus tremula x tremuloides
atggcaatagattgcatcaaaaccatgccatccataaccacccctcaccaccacccaaaagatcaggatcaatgcaaagatgatggcaagtcttttgtctttgatgcacaagttcttcgacaccagacaaacataccccagcagttcatttggccagacaatgaaaagcctaatattaatgcacctgaactccaagtcccgcttgtagacttgggtgatttcctctctggtaaccctgttgctgcagtggaagcttcaaggcttgttggtgaagcatgtaaaaaacatggtttctttctagttgttaatcatggagttgataaaacactcattgcccatgctcataattacgttgacaccttcttcaaattgccactttctgagaaacaaaaggcccagagaaagattggcgagtcctgtggatatgctagcagctttactggcaggttttcctctaaacttccatggaaagaaacgctttcttttcgctacaaagctgaggagaattcatcaaaacacatcgaggaatactttcacaacagaatgggggaagatttcgctgagttcgggacggtgtatcaggactactgtgaggccatgagcactttgtcactagggatcatggagctattaggaatgagcctcggtgtgagcagagaacatttcagggaattctttaacgagaatgattcaataatgaggctcaactactaccctccatgccaaaaacctgaccttactttaggcaccggtcctcattgtgatccaacttccttaaccatcctccatcaggaccaagtgggcggtcttcaagtgtttgtggacaacgaatggcgttcgattaaccccaattttgacgcttttgttgttaacattggtgacaccttcatggctctatcgaatggtatatacaagagctgtttgcaccgagcagtggtgaacagccaaacaccaagaaaatctctcgctttctttctgtgtccaaagaatgacaagatggtaactccaccacatgaattagtggacacatgcaatccgagaatatatccagatttcacatggccgatgttgctcgaattcacacaaaagcattaccgagctgacatgaagacacttgaggtgttcacaaactggcttcatcaacgaagttttagctga

SEQ ID NO: 18 - protein, 385 aa, Eucalyptus urophylla x grandis
MAVDCLTSKTSPAMPPQHKDEAREDKKHLVFDASVIRHQPDIPKQFIWPDEEKPCANAPDLAVPLIDLDGFLSKDPSASEEASRLVGDACQKHGFFLVVNHGVDAGLISDAHKYMDKFFRLPLSEKQRAQRKLGEHCGYASSFTGRFSSKLPWKETLSFGYSAEKSSANVVEDYFKNTMGEEFEQSGRVYQDYCEAMSRLSLGIMELLGMSLGIGRDHFREFFESNDSIMRLNYYPPCQKPDLTLGTGPHCDPTSLTILHQDQVGGLQVFVDNEWRSISPNFNAFVVNIGDTFMALSNGLYKSCLHRAVVNSRTPRKSLAFFLCPRSDKVVRPPSELVAMSCPRAYPDFTWPVLLEFTQKHYRADMNTLRAFTNWLQQRTSEPVR

SEQ ID NO: 19 - DNA, 1158 bp, Eucalyptus urophylla x grandis
atggcagttgattgcctcacaagtaaaacctcaccagccatgcctccgcagcacaaagatgaagccagagaggataaaaaacatctagtttttgacgcctcggtgatccggcaccaacccgacatcccgaaacagttcatttggcctgacgaggaaaagccgtgtgcgaacgccccggatctcgccgtcccgctcatcgacttggacgggttcctctccaaagacccgagtgcctccgaggaggcatcgaggctcgtgggggatgcgtgccagaagcacggcttcttccttgtcgtcaatcacggcgtggatgctggcctcatatcggacgctcacaagtacatggacaaattcttcaggttaccgctcagcgagaagcagagggctcagaggaagctcggtgagcattgtggatatgccagcagtttcactggcaggttctcttccaagctcccatggaaagaaacgctttccttcggctactccgccgagaaaagctcggccaatgtcgtggaagactacttcaagaacaccatgggcgaagagtttgagcagtccgggagggtgtaccaggactattgtgaggccatgagcagactgtctctaggaataatggagctgctaggaatgagcctaggaatcggcagagaccatttcagggagttcttcgaaagcaacgattcgatcatgaggctcaactactaccctccgtgccagaagccggacctcaccctaggaaccggtccccactgcgacccgacatccttaaccatcctacaccaggaccaagttggcgggctccaagtgttcgtcgacaacgagtggcgttccatcagcccgaacttcaacgcgttcgtcgtcaacatcggcgacactttcatggctctatcaaacgggctatacaagagctgtttgcacagagcagtggtcaacagccgaactccgaggaagtccctcgccttcttcttgtgccccaggagcgacaaagtggtgagaccaccgagtgagctagtcgcaatgtcctgtccgagagcgtacccggacttcacatggccggtgctcctcgagttcactcagaagcattacagggccgacatgaacacgctccgagcattcaccaactggcttcaacagagaacatctgaaccagttcggtga

SEQ ID NO: 20 - DNA, 1799 bp, Populus tremula x tremuloides
gcgagcctgtcaaaacaaaccattgacaccaatcttggatagaataaatgtgggaagaggaaattgaaaaaaagagagaagaaattggcaccgtggactgctaataggtctgcatctgaaacaatggattgcctaccccaattagtttttgttcatcatgtactgtgttctcaaatcaagatcatacttgggaaagtacaattgacggtttggaattaacggtgcagtgttgatctcatgttcttaaaattcactcagtcatttcaaacatctaacatgatcacacctatgaactgtcaacaacgaaaagttgataaaagtaattttgcctttcaattctcttttgaaaatttgccagggaatgctattttgagccgttggcgtttgagaaggttgaaattgcctatcttcaaaagatgctggacccgttttctatttaaaaaaacggttcgaatttagagagaaacggctcttttggagtcacattaatgtatttttagaagttgggtaaaagttaatttttaaaaatattttttatttaaaaatatattaattttttaaaaaacataattaaatcacagtcataaacgcgtccgtagtgagtataatctgcagggcggcattttttataataaaggtaatttaaatcaggtcttctttccggtgacttaacactgattttactccgactccaacttaattccgtagataattatgtaaaataaataaatgacgcttaaagttaagctttgtcccctaaactccttaatttaatttaatttctccttcttcttattaactcgactgagttatcgatactacgtgcaatatattatctccactaatggatattatttatgtagaaccaaaaaataaaaaaataaacaattacttttgatattaattaaattccaatatcaggaattgagtaaatagatattatttacttttgagattatgatttttttttaaatcatcgatgaggtatttaatcaggaaagatttaaagcgaaatccgaagcaaacatctttcaactccctgaaacgattttgttgctgtatttattgttattattataactattcatgaattaaaaaaaaaaaaagagtctcttgtgagtcccaaacaaaatatatgtgcccattaagttaaagaaattaaactaaaagccaaaacacacctttgaatttgagtaaatagatattattttttaaaatattatttatttaaaaatataataaaataaattttttatattttaaaatttatttttgatattactaacaaaataataaaaaaattaatttaaaactaaaaatatttacaataacattattgaaatacaatgtgcatggtgtagaatatgtagatgtatgtatacagtcagtttgatattatgataatatttgttttttaaagtatttttattttgaaatatattgaaataatatatattttattattttaaaataaaaaaataatttaaaataaaaaataaaaaaatttaaattttttgaaagcaaaattaaatctcgtaggcactagaaaacgtcgtaaaatatatttttaactggaatctagcacagcagcagcaacctccctagttctcaaagtgtctttccatttgaaaaaccattaaataaatcgtttctcctcctcctcctcctccctaccatcccaacacacacacaaaaagaagaaaaaattcaaacttcaaactcaagctcagatcccatcctctctcaaacgccaccttttcctccagatccagcctcgatctaccaaatcaccgtcaccg

SEQ ID NO: 21 - DNA, 2000 bp, Populus trichocarpa
gtgttttcccatatgattttttatgatttttttcaaaattatctttgtcgatttttgtttaatattgagttggttaagaattataattataatgaagctaaatcatatgggaaaagcgttgcagattttctcacaaaacactatgaattgctacagtatttctctaaatggttttttattttattttatcggggaaagcattgtagttttcctcacaaaacattatcaattgctacagtgttttttttcataggtattttcattccaaaattatctttgttgatttttttttaatattaagttggtaaagaatttagctttataattctttttgctttttattaacagaaaatctaaatcatgtggtgaaaacactgtgtctttcctcccaaaacactgtgaattgctacagatcattttgttaagtctctaagttttttatcaccaatataactttatttccatcataaaatattaattctatcatacttttaatttttattatttatctaatactgatacataattataacattattaatacttgtgtttataaattcatggcaacatgcgcgcatgccatctcatgataataaaatacaggagttcttaaggtacattggttttgccacttgcatgacgttgggtttctgctcagaacgctggctgacctgttgccaagaatctttgtgagcatgccatggcccaaattgaatataacccgagttatatatatatatatatatatatatgggtttgaccaataaacaatttcttcgcaaatgtttaaaagaattataattatctccttttaggatctctgttatgctagtttgaaaatgtaattgtgattattttttaaaaaaattttatttaaaaatatattaaaataatatttttttttatttttttaaaattatttttgacatcagcacatcaaaatgatttgaaaacattaaaaaaatattaatttgaaataaaaaaataaaaaaatttattttttttttaaaatgcttttacaatacaaaaacaaatcaaatctaaacactaatactttcattaaattggtataagtcacggcaactttcactcaatagatctttcatgtttttatatataaaaaaatttagacactaatactaacttgattttataattaagttatttcttatattaatttcaacttaaaactgttttaattcgtcgtcaatgacttctgttaaaatcatccaacaagaattattattattattattattatttaaaataatgttgtttctgttttaaaaagtataattttggtaaaaaaaatataaaagttttaaaaaattcacataaaagttcttaattatactcttttaatttaaaagtgaaattaaaaaaaaatcatttgacaataaaatcgaaaggtgggtatttttgagatctacttttcaagtttctagcaacggaaaaccgtgaaacttgtgtggcagcaaattcaggcttgacctcgcaggctgctgatgtcagcagagagctcatgcaattagtccacgacaagagccttccgttgctgggcccaacaactttgagcccatcggacatactccgaatattcccacttccagaacattaagactcccacggtcattaaggaaagtgcgctataccttcctcttacgcctctccctcatagataaacaaccagtcaaccacccaagactgaatccaaatgccacaaaatcgcgtgaatttcctttttaaaatcagcggctaatacaacggcttttcaaaattccaatcagcggcttaaaaccaagctaattgtttctttcgttttgcccaagaaaaaaaaaaccgctcacgtgacctaaccccaccacgcacctactcttctttatatgaccctcaattagcagccactctcctcactaaaattatctctgtttaagcttatccctcaaatctcccctcttaattctaaatctctcctctgtgtcccgttactatcaacaaca

SEQ ID NO: 22 - DNA, 2000 bp, Populus trichocarpa
agttcgcgtctcattacaagcgtggggtgaagatgatcaattacatgtaactttggttcttgagagcaagattcttgctggatcaagttgtaaacttttcttcaaacttcaaattagtaatctaagagacaattccacatcaaatagtaattaacaggacatggatcaaatgttaaggaaaaaataattaaaagggttatcctaaacttttaaaggggttgatgcaaattttaaaggaaaaaaatggtcgttggtgccaaactagaggtgcatttagtgcacccttcgcggggggctccaaaatctttcaaacactatcatagaaggcgatgtttggtgattaaatgtccccacacttgtcgtccacttgttagggcatgcaaaaaaattttaaaattattttttaaaatacaaaaatacccctgagttatcttgatattaaaaaactatgatgaaaagacaaaaaaaaaacttgaaaaatagttttgttaatttcaaacccaagtataattaagtaatttaacttcaatagaaaattaaaaaagacataaaatcctctgggtttaagcccttttttttttgttccgggatatttaaataacttaactgttttttttttttaaaagactaatctaccataaaataatttgaaaatgacgttgaagaggatatgtatttttttgtttcgacagtaagaatattataccgagctagcttgtgcgtcaacagaaattcaaatttttttagaatgattttaaaaatatattcttcacattgacatgataaattaaaaataataataattactaatactaagatttagatttaaaaaaaataaaagaactttcttaattaccgaacaatttcataggaggtaatggtttacagtttacaatacggcgatgatttggtttcaaggacagtcaaaatcggaaattgccccctgacgaccccaggggaagaaaacaataattacatgaaaagatgagtggtcaccaccactaccaccaccttgtaaaaatgtatgacgacccaaaacaaaaacgccaactacaccattaacaccacaaaacccattttaggcaactaatccttgcaccaggaagagggagatgtattttcatgcctcttctaaatacaatcatggtaattacattacattaccttacccatccccctctctctgtgcattaccgtaaggttataaagacagctcaatgcaaaaacaaagagagagagggggagaaaacgacttgttattattcactagaatggcattggttacaatcagaaaatcatacaccacacccaataacccaattgagttatagaagagagagagaaagggactctgagagagttgcaggttttatagcagaggtctctgacagtaagagagtatatattttaactttatctatcaaataattctctcttactctctctctctatataggagctagctgcttgttctctttatgtttctctctttcacacttgtatctttttggtactgatgaatactcaatgagtctactgtttttcttagagagatagagagacttgtgtgttgccagaacgatgtgatggaacgatataaagccaagccaagccagactgagagctagtcatgttataactacattacataagccccaaaagcaaaaacttattcaaaaagaaaatcataaccccatttgcataaacccttctctttctctgaaaaccttgtattttcatttcattgtcggcgacttcttctcaatgagagcttaaaagggtttcttctttctccaagttttaagtgacgggaatggttattttggctgcctagagtggttaagtttggaggaggggggtttttgaacgatggttgttctcaagtgagcaaattttggattctcttttttggaagagcagtgaagtgatggcatttcgtttggaacttgtcctttttgctcttcttctctctttggttgttgattctgtgcattctgatg

SEQ ID NO: 23 - DNA, 2500 bp, Populus trichocarpa
aacatcttggatttgtttactcatcaattggccccttatgtgggaactgataaaaaaactatcaatttctaaaacttatttttgacactttcatgtcattgcatgacacacaaccaaaacactcatgggttaagatgcaagataaatctacttgttatcacgaacatttctacccagctagactttcaacaactcacaagttagatgcaacttctcacccaaaccgtattggtaacccaaggtcaaaataagaccttaccacccccaacaagtcttggtaactatgacaacacacttgatcccatcaccatttccactggccgctcccaacgcccatagcccattgcactacccataagagcctgggtagaatgacctccaagtgagtattctgcctccatactcgtcaagtccctccaggggtcgctcgcgctattgtgatacccgtgcacatagccaggactccatattgctcccctaactaccgtaggatggaccttggttaccaatctcgttgtttcatgccaaactttttaggccatactcgtcaagaccatgaatgatgaaattgatgctattgaaaaaaaacaacacttgaaaatcaatgaagcttctaaaaagacaaaaaaccattgatgtaaaatggatttttaaaacaaaattgaaggggaatgataggttgacaagtataaaacatgcttggtaactaaaggtaacaagcaagagtacgatgtcgattactaagaagtttttgttttagttgccgggtatgatacaataatattttttctttggtaactcaaaatccatggtttatgttgaatgtaaaaccaatatttttacatgaagaccttggttataagagagttcttcatcattaaactgattgttcaagaacatcgatataaaggagaaatagttagatattagttgaaatcctattccaagtagaaattcttgatagaaaaggatttgtgtctaaaatccttattgaattagttaattataaatcttattggattatgattgttagaggttgcatcctataaatataaaacttttaccagattttctgtccttctgtagtggttgaaaaatgttgcagggccatgacgcacgctatttttgaaagaaaaatatacaggagaaatagacatgttgaaaacaaagaaaattcgaggcacacgcacttggtttttatagttttgtactgggcacatgggttaaaatctttcaggcctaaccatacttggttttatttatttatttatttatttggctctaatttgtttttacattttacaataaaatcaactgtaaataaatttattcagacattctcttttctagaaaaaataacgtgtcttaatactcgaatatcccttcttaaaactcacatgtacatccaatttatttttattatatgagaaaaaaatagtattatgttccaagcttacacttgaaaaaaaacaacgaaaaggataccgtttgattacgaacgcagcatcggaaaataaaataagaatttcgactttatcctcccggaggggcctttgatcatcattcccggaatcttttaaatatatcacagacccacttccccaccttttgccgtggaagcaggtgctcactccacaccgaatgacccaaagtcgttaaaaaagcaaatggagtctctgttctttttcgcatttatattcaaatccgaaaagaaaaaggtttaattttacaccacgcgctctatatagcgtggcacgtgacaatcgcgttttgttttttttacatcatcttaagtgattttcttttccttttcaatgaaaatatttaccctttcttcatttgtaatttcaagagattatatattgcctgcactttaacatgacgtcaatgaaaccgttaatccctttcaaaataaattccgacatctgtttttttatccaaagcattctgtatgtatcttttaaatttttgaaaaaaatggttttttataccaacagctcccttttttttaatattttatttattctggtcggaactaatttctctgattgaacttcccttttaactttttttttgtaattataaattaactttattaagctaaaaaacaaatattttagaggtaattatgagtacgatagcgttgttatttttaaagtatttttacttataaattcatttaaataatactttttattttttaaaatttatttttgatattaacatatcaaaaataatttataaataaaaaaattaattttaaataaaaaaattaaaattttatcgaaactcacaccagcaccgaacgggccgaagttgctcacttcacctaatgttttatagataaacaaaaagtcacgctagcaattcacagggaacagtcaccaccaatatcatgctgccagatgaataaagcagaaagtcaaaaatggtcacatcgatgacccgcataagaagctttcaatccaaatgaacggtgggtgatggggctgtgagagca

SEQ ID NO: 24 - DNA, 2700 bp, Populus trichocarpa
aacatgcctgtaataataataatttaaaaaaattgatattacggaagttttgctattgtgcgctgaaacggtggtcttaatacaagctacaagcggactcctcgttgacttaggttttgatttatagtattaagtcaagcaatagattaattaggattattattattattagcatgggcttatgtttagagtaattgtgttaagagcaacagggaccacgtacgtgcctgatagtgtcacgcttcccaataacatgaaaccgccgtagccctcaatcactctgactgcaggtggcatatgcaacctttataaaaatcgagagaaaagttgacttgttaaagtcagagatatccatggctgtgattttgcccttcaatacccttaaataacaggcttgttgtatctgctatgttttttttttttttttaaaaaaatggattcacgtcatgtgtgtttaaacaggagagcgcatgattacaggaggaggagtgataatcaacccacaaaactagaaagcagcaaaacgacggaatgtggcgcatttgaagcgctgaaatcgagcgtacaccgttgcctctttttttttttttttgtctatatctcttcctctgtaaaccccattaatttaatgcgcctctcacttcccactctgtagacactgctacctactctctctactctcttagctctgtctttctctgcaaacactgtgctgtcagttgctgttgtggagatatataatggtggtggctgtgatggaggaggtgcaccggagtagctgaccctaggacattccttattatataaaaaaaaaaggaaaatcttgtttttgccctttgtcattcgcgtgccccctttttaagcctagtgtcagtcgtcagatctctctctttctacatgtttaagtttgttagtgtttttctcgaaaaattaattttctttgcagtctcaagtctaatctctcatttctgtctttgctctctcgtattctgttccgttcctgctcgttcctctctctctagctaggcaggcatctttctccctctcctccagggtttgttttagcccttttcaattcccattttatataaattaacagtattacattcgagatgcgtcgaaaaagaagttcggtttcaaccacttgaagaagaatgtacgtgaccatgttcctctttaatatagtttttcttttcctgctatcccttgcttcttccttccaaataagaggtttggttgctggattgtttttttctgtatgtatatatatatatatctctggatctaatgactgcagcttttgctttctcgtttattattagagtagtaaaaaaaccgaagtaacggtaaagattttcacacaaaaattgttttgtcagcccggtaataaagtatagttgcttgttgtgttttttttttttttttttgagattgtgttgtttcgatgaagagtgctattgttaatattttataacattgttgcagtagcttaaacgattatctttttttcagatgcctctgatttgtggctaatatataagttgttttcttagatttttcagaaatattgatatttgaaacacaaccattagatttggaggacgttgatagaaatttggtttttttttttcttaagctgcaggtctatgtatagttttgtattttccgttgtggcttgaaaaaataaataaataaaagttatgatatcaagaacaatggatcatacggttaacaagtttcgctttttgcaccttatttgttggtaattgattatgggatgctcacgaccattattagtattttgtgcagcaaacaaatccacaaagatgcatttgtcataatatgcccttttttttatgctgaagtttatgattgcttgaggaattctggtagatataagtgaagtttcactgaattcttttgggtttacaaatcattgctatacacgtttttgggagttacatatgatatggtaggggaaacaaggatttgatgagattgtgtatttggatgaatttctcaagtattagaccacaccacatgctgtttcgttgctgatgcctatggcccaataccaatggtgaattattggtgctgagcatttctaaaagtggagctacttggtgaattattgcttgacatcatttgctgtttgggctggaattctatcatcataacccagtaaattgctcaatctgttatcggttgtaattgcttacttgtgcctgacttatgacatgataagaaattcatcttgcttattccgtgattgtttgttgccaatgatagaagcaatgtgtttgttgtgtgttttaggattattttattttttattaattctatgtctttttatggatactgctactaaacttgttcaggtgtgtatatatatcatacacatgaaggcgagcttttcattactccgtttgtacattcttgatgcaggttatggcccttgttcgcgggtccaaggttgataattttgttgggatccttatgttaatttctgcagtgcggacctacttatttagatgttatatattcatcttgggctaaaccctgcttttcttttgatgtggaatagtagagtaattatttacatgttttgccaaggtgaaggagattgatgccatgttctgctatgtttcagatacattatgaagagttcaaggcaatggaaagtggtgtctacacctagaa

SEQ ID NO: 25 - DNA, 2500 bp, Populus trichocarpa
ctctataattgtagaaatcttagattattgaacctgaataccatcaacaaacacaataaaccaagaaaaattacaatactagtaaagaacttaaactttttgcgtttgacatataattgttctttccttaaagtgccaaaaatagcccttaaatcctcagaatataactataaatcaggatgtcgttaaaataaaccacaacaaatttgcctataaatggtctcaatttctaactcataaacctcatgaaagtgctgagagtattgcacatatcatatagcataaccatccattcatataaaccatattgtgttttaaaggctatcttttactcatcctcgacttgattttgatataatggtggtcattcttgagatcaatcttggaaaacacctttgaacctgccagttagtcaaacatgtcatctagttatgatattgagtactagtattttattgtgacctgttaatggctcaaccccatactcatatacgtcatgaaccattatttttagggaataagagagctaaaactttacatgagctcatactttcttaaatatactccttttttagcaacttcaccatttgtttcaacaaattttcttcttttttaaggctagtctataagttagtatattcaatagactaaaacttagtactagatcaatatggtgttgtatctttctcatgaaaggtaatttctagggcaactcttttgacatcaaatctagaaattttgttagcataccatgcatttaattgtaaatgtcattaacattgtcctctttcccaactgcatttcaacttgtagtatcaacttctctcaacctcgttcaaccaatttatgaattcctctatttgtaaaattcctactaactcaaggattttgatatgaaatccaaaatccttcaatatgttctctttttgatcaagaggttgtctcctctgattctgcctttggtctttagtgttggctagggacccatggttgttcttctagtcttggtctcccaggtccctcttctctaacctgccgttcaactctgcaatctgcctccgtatgtcctcaatgatgaccttctaaatgtttgtctctaatgaggtaacctcctaatcccctccttgatcatgccacacttcgtaacctcatcctttcatggttattaatagcaaacaacaaataaataagtatccacaagattgtaacacaacaaacaatgaacctgagatcattaggctctgataccaattaatactagcaaaatcacaatagacaacaacaagcataaaaaaaatagaatttttgatgagtgaaaaggctgcagatataagccttaaattcataatttattattctaaaataaagtctaataatatctacctcctaagaggttatataagatatttaaatatgtcagaaaaccctatctctattaggatacaaatcaaaatttgaaaaagaaaaattaacataaaatccaaaataaaataataaatccaaaaaaatcaagaaaaaattacaaaattggcccaaataacatcttctagatctaattttgggagatccattagtttgatgaacgacaaatcatttttttatatgaacatggccgttagaattatctaatcaaatttaaaatcgattcaacgatcagatcttctgttatgatttgttttagttattattttggtctgacataaccaagctttttttttgacctagacttttctcaattatccataaaaacatttattagatcatccatatagtttgtttgggacaaaacctcattcaaatgctcaaaatttcagcttttacaatctcgactacattgtctcattacatatatgtttagatgaagttttattatatatatattttttttttgcatgtacatcattttcaatttaagaaactcctttgtcattgtgattacaatttaattttatatacaaattatgatgatttatttcttttaatttcaaagttttttgttttatatttaaatttttaaatatagtcgaatcgccgatgacaatacataataagtgtactttctcctctcatcttgcaaaaactagccacatcggatataggccacccaatctgataatggggttgggtaagggttaggttttccattgattttaatatttattgaccttttaattatataaacatttgttaaaaaaaatgaagaaatttagttgtactaataataacgaaaatacaaaaataatattggtagcgaagtaatagaggaaaggtaataagaaattaataacgaaaaaaaaaaaaaggacaggcgtgaaagggttctcagataaaatccttcccttccctactgtatttgtctctctctattactccatctctcggtagggcctaaaaagagagaaagatatgcaatatatacaaagagaagcttgagagattttaccttccttttcattttcatggttttctataatgagaagaggattgaatctcttcttct

SEQ ID NO: 26 - DNA, 2101 bp, Populus trichocarpa
gcggtcgggagagagggagaaagggtcatgtcagccaggcagccaaatctttatacttgaattcttgtttaccggaaatattgtatgtaatgaattttatccacaaggaaattttttttatgggaattatagccttttagatgcatgaaagttaccctagagtcatgccctaaagaatttttgttacatcgatcacatgcactgagaatgttttccactgctatcatgtgggaaatgactttcagtccctcactctatggtctatgcctatgaatgtctcttgtctaactgcttccagaaaagaggtggttttttgaggaaaaaatacgtcctccgagctgcatattccaccttaatatcctcacaaaatcagtgcaaattcacaatagatcatgactcatcaccacacgactaggtcaagataagaagttgtggggatgtttttccaaaccaatacaaatgtgtttgaaagttttgaagaaatagcatcgaggataaaaggattttgaaaaatagcacaataaacaaaaaaactacaataaaaatgaggtatttaaacaaaacgtagttgagaattgtgatatttaaatgaagatacctttatactaaatgctatttaaaaaagaaaaaaaaaccagtagtttctgtgatttaaaaaaaaatcgcatgcaagaatcacagattttctagctgtgcttcttttcaaaaaaacgtttttatacgtactagctagtcaaatttttaaaaaagctctgctaatttcttataataaaattgaaggtttcaccacacaattattgaaaacaaatttagtgcataaacgacatatttgaagtcaattttttagcaattactaccagaatcttgtccattagaggaaaaacatgctcagctcagtcgaggtttcctttcaacagtactcaataaactctggtctgtccacactgcccccctaagaaaatcacagctacgaagggcaatcatataagaaaaatttaccatttggcaaatgtttgcgataatgattcatgacaatgataattctgattaatcaagcacatctgctgtttatgtctgatcacaatttctttgcccacccaatcacacgtgctatatggaatcacttattgacagttcaaagtgcatgatgcccgtgattgcagtcagtcgaaatccttctgcaatcctccagtttcactgagaaactaatcataatgcacatgaccagaactattattctctccaaatgttattatacgtgcttatcatcaactaatcagtggtctgtcagataatacataaatatttaggctatggcttgtttacgcagttcatatctccgagtgttatttatagtctcgcttcgcttgaaaaattattaaaaaaaaattatctttttcaattcatacggagttgaaaatgccgcagtaccaaacaaacaaacacggaacaggacatgagaaggggggataaatgggtggggtttggtggtttttatagtttatactatgtgggaaattggagctacagatcccagagaagaggaattggatttggatatggcatgcactactgaaataaatatatattagggttatttatgttttaatttttcaacgttctctaccagttaaaagacgacggcattcaccaatatttatccgtatcttgaaaagcaaagaagttccattttcaagtttagggattggaggaatctttttcatcctctccctccctctctccctctctttactttatactgtaatgaaaaagcttgagagagaaccagagaggccaccgaaaagcatggacaccttatcatgaaagcaagaaaaaaacgaaccgatttgacgggccaacccacacgcataaacacacagttactcactcactaacacacccacacgctcgatcgatcacacatttgtgtcactcactgtgtgttaatttttgggtgtctttccttttgttaaaaatatggaaatcataagtcaacctcccctcatgatttggtcttggtttcttctaagcaccacctcatatcccactttctttagcaccctctatataattaccactccctttcattcagtccttcacc

SEQ ID NO: 27 - DNA, 600 bp, Populus trichocarpa
ttttaatgcacttgatgacatattttcaagctattaaaatttaaaaaataaccgctatcttcctttccggcaccgtccgagcccacaacactacaattcgttgcctgccacttcatccaaatgtgatgtgaacttaaatgagtagagcccatatgtatgagcttaactaagatcatctggtcaatagccgagcaccacaagggtggtcaggtgcatcttaaaggggcttgaaagagagagtagaagccgaagctcgactcggcgataacctcaaatgaactagtatcacacatccatgtcctcccgaggatgagataaggtcctggagcttcttccacgtggcaaatccattgtggtgcctaacgataaggctagcattccaagaatttccaccctcatgtggatagaatggtgtcatgtcatcacttatcatatccaacggtcacattttgctcattatcaaccacgatagcttgtgtccataggatatgagatagacaagttgaggcattatatatacggtgtggcaggagtacttcaatcttacaatactagcaaaaccacatatagagggtgtaatagctaagtagcctgtaagag

SEQ ID NO: 28 - DNA, 1800 bp, Populus trichocarpa
taaaacttgatctctaataaatttaatattaaaagataaattaaaaaaaaaactaaattaaaaaaatatatcaagttatcaaattaataacttgaattataagattacatgattctatccaaaacaaattaaaataaaatataatgcaaaacataactcttgctttatccattaccatgacatcatatgtgacaattctccattatctagtaaatctaataatgaattataaatttttaaaaaactaaattataaaaaataaaaaaaattaataaattcaatatttcaacaaaccttgttttatgaggatatatatatatatttgttaaatcaaacatgtacaaattcataaaacaaaggacataatcaataagagctagcagggaagaaacggagaggagttgtggtggtagcattgagtgagaaaaaaataataatggtctgttatggtatgagcccacatgctctttacagatggccatgagaagcttctattggccaattgaaagtttgaaacatctagaccatttcctttgttttcccctgaatatgtagcattgggtttcataaatacactggaccggaccatcaaagccttgtctggtggactaaattgcatcatttaaacatttaaagggcctgatgcataattttgactgtgacacttaacaggcctacacggtccaaactcgagggacctgaacatctatggggccttcagctagtttagggtttcattttcgataatggaagctccatcccttttctaataattcaagtttaaagtttctcctaaaagctccactgtgcaattgaagattgagtgtaaaagacgctccaagagtcattttcgatcacattgacatgccaatcgaaagtgttcttttttattattattattaatgtgaatgtctggactaatttgcgcgtattttgattaattctacgagccatgaagttaacgaccatgtaaacttccaatgaccctgaactttatgagattcgaattggtaacttctagaaaacaaacttaaaacctgatcagataagctacacctctcgagattgtgttcttcttctaagcagtgggtgccaaaaaaatcaacccatgcatgcaatctggtatcaatttcttagaaaaaattgattttgaaaaaagaaggttacacataatttaggggcatggttcactttcattctttttaatgcacttgatgacatattttcaagctattaaaatttaaaaaataaccgctatcttcctttccggcaccgtccgagcccacaacactacaattcgttgcctgccacttcatccaaatgtgatgtgaacttaaatgagtagagcccatatgtatgagcttaactaagatcatctggtcaatagccgagcaccacaagggtggtcaggtgcatcttaaaggggcttgaaagagagagtagaagccgaagctcgactcggcgataacctcaaatgaactagtatcacacatccatgtcctcccgaggatgagataaggtcctggagcttcttccacgtggcaaatccattgtggtgcctaacgataaggctagcattccaagaatttccaccctcatgtggatagaatggtgtcatgtcatcacttatcatatccaacggtcacattttgctcattatcaaccacgatagcttgtgtccataggatatgagatagacaagttgaggcattatatatacggtgtggcaggagtacttcaatcttacaatactagcaaaaccacatatagagggtgtaatagctaagtagcctgtaagag

SEQ ID NO: 29 - DNA, 2500 bp, Populus trichocarpa
ttttttgactcgataaataataaatataaactcgatttaatttgatgagtactcactatcatctttagccacggtgaatccagcttgccacttgtttgatttaaataaaacagtaacaagattcgttgctccacgtgcaaatgctaaatcgaaccagctagatttcatcgaccttgtaaatccaatgcacgtggctcacccatggcgggaaaacacaactccggtggtgaataataatagcacgggaagaaaagcagtggaaaaaaatggtcactgaagcctatatttcttctttttttcttgctcttcgagcaactaggatattaatttttattaatctttgaaaaaataataataataaatatcttataaagcttaaagaatcgtgaatattgttaaaaaaaatcactataattgatgtaatgcttatctaacccttgaaaaaataacaatgatgttgatactcttattatcatagttttaaaactcggtctagcaggttaatcctgaatccgacagatccggggttaaaatcgagccgagttgagaaaaaaatagagaaagaaaaaacccggtgtgacccggtcaaaaatccggatacaacctgttgacttttgtttttttatttaactaaaaaattgacccggacgacctggtcaaaaccagaacccgaaccttgaaccgggccggattttaaaaaatatggttcttgtattagttttctcctcctttaatatttttaatttcttttagtgataaagattcagacaacataggatgacgattatccttttatatatatatatatatatatatctgacatttttacatgagaaaaataacttaaagtatagggacttaaaaatgccattaaactaacggaaaacagcaggagagtggatcgagagacaagttttattacagtgctcttttcaatcgaattcgtttagaatttcaagctgtgtatcataaaaatttaagattttattttatttaagattaattttttatattattaaatcgttttaatatattaatataaaaatacattattaaaaactaaaaatatattattttaatatatttttaaacaaaaaaacattttaaaaaccacctttacaaccttaccaaaaactcataaaagcaacctcggcgcgtgcacagtctaccgttacagattctgacagtctccgttcgaatacggtcaacgcttttgacgctttccagaaccacacttacatctgcgcgtgtcaagcacatacacggatctgtgagaacttgtaaagcacgcgttttcttgaaaggcgacgttagaaatcggttttggtatggtcatcatatgcttactcgcaggttatctcttttagtttttagatttttatttttatttttctcgggaaatatcggtgacgcccgcccttcgagtggcggttagtttttcctatccccttgatttataatttgtaacttttattatcattgttggatttatcttattataaataaaaaaaataatgatattgataatattataattcaagtttcagtatatttattatattttttatttctctacttttttgtatatttaatatttttaaagtatattattattctaaatatcaacaaattataaaaatcatgttttttaattatgatatatataaactgtgttattttatcaatacttttaaaatatatgttaaaaataaaaaataaccctctttccattaactatccatacgaaagggaaaaaaaaatctatcaaggatttaggatttaattctaaaaacaagttatgaataattatttttttcattttacaacctttgttgtttaattattctttcaaaatatattaacaattataaaaaaaaactaaaaaagaagtgatttttagtgtatttctccacacaaaatattatttattataataatgtttttatttttttatcatttgatatttttttatttagtattttttttaatttcatcatttaatattaaatttattttttattgagttattttattttcatgacatgaattatgagttttgcatattatattaactaactcagtttttttaattaatttttatttttttattttattttttaatattaatttgattaataaaaaatctaaataataataaatttttacttgtataatactatttgctaaaatattatgtttttttcaaataaataaatttaacgaaaatacaagaaaataatagcaaggtaataacggaaagcgcagtgagaaaaagaaaagaaaagaaaagggcaaaaaaagacaggcgtgaagagttctcagataaaaccccttcactcgcttactctatttgtctctctccattacagtagggcctcatagagagagaaagatatgcaatatatgcaaagagaagcttgagagatttttatctccctttttcatttttgatggtgttctataatgagaagaggattaaatctcttcttctctgaagaag

SEQ ID NO: 30 - DNA, 2101 bp, Populus trichocarpa
atgctcaacctttttaattttttaaaaaaaatctctttcactacttttcttgttttatataattcaagaattggttgctatacgaagatccaagttgttgatgataaaaagaaaggaaagaaaagaaatccaagttgtcggttctttaaattgtgcaaggaaagcaagagcagataagcttaaatgttaagaggaaagccaaaggtctgcatgtcgtctgtgtataaacccgtcaagagtttagaatgagttgttcagcaacccaaatccatatgtcaggccaaaaacattgttagctgaaggatcttattgtactacattgcaaatatgtggtcgagagagagaggttcacatcaactcggtcgccaaatctttatcttagaagcctttgtttaccggaaaaaacaaaatatagtgaattttgtccataggtaaaaatcatatggataatatagccagctagatgcatgaaagttacactagagtcatagcctgaagaatttttgttacattagttaatcaaatgtactcgtaatgtttttaacagcttattaatttaacatgttttttaaaatcctttttaaaatttgttttatataaaaagaatattaaattaatatttttcactgttatcacgtggaaaatggctttatccctcattttaaacatgttttttaatatgctttttaaattagttttttattaaaaaaatattaaattgattttttaagatgttttttgatttgttaatataaaaaatatattttaatacatttttatttcacattaatatcccccacaaaatctgtgaaaattaataatggacgactccccaaccaagtcaagacacgaggtctctccacacaattactggaaacaaatttaatgcgtttaggacatgtatttgaagtgcaatcgcttccaagaacgtaaccttgtccataagaggaaaaacatgctcagctcagtcgagattcctataaatagtcaatgaactctggtctgtccacattggcacccttaattactaaaccactagagaaagttacagccacacaagggcaatcatatatatataataattaagaacataaatttaccatttggcaaatgtttttttatagtgattgatgataatccagattaatcaagcacgtctgctgttaatgtctcccaattcatttaccaaatcacacgtgctacgcagaatcacttattgacagtccaaagtgcatgatggccgtgattgcagtcagtgagatccttctgcgatgctctagtttcactcaacaactaattataacgcacagaatcagaattattattcaccccaatgttataatacgtacttatcatcaactaatcagtggtttatgagataatacattatatagttagtgtaataagacacgataaggtactgccatagaattaagaaaaaggggaataagaggtggggttcggaggtttttataatttagagaggttattctacgtgggaaactggagctagctacagctctcgaagaagaggagttggatttggatatggtatgctgctactacattcaaatagatatattgtgattatatatgctttgttttttcaacattctctaccacttcgagacgacggcattcactcgctggcttctctcagtcaccaatatttttccgtatcctgaaaagcaaagaagttccattttcaagttttgggattggaggaatctttttcattctctccctctctactttatactataataacaaaaaaaaaagcttgagagagaaccgaagaggccaccgaaaaacatgggacaccttatcatgaaagcaagaaaaaaaacgacccgattagacgggccaacccacacgcatatacacacagttactcacacacccacacgctcgatcgatcacacatggtctctctccctctgtgttaaatttgtgtgtgcctcgttctttcttttaaaaatggaaatcataagtcaacctcccctaatgatttggtcttgggttcttctaagcaccacctcatatcccactttctttagcatcctctatataattaccactccctatcattcagtccttcgcc

SEQ ID NO: 31 - DNA, 600 bp, Populus trichocarpa
aaatctagtgaatccagccaaacctaattggttaactctaaatttttttattttaaatatgaaacaatattatttttaaaaaatcttattgacccaaattgacattaactttttttttatatatatatatatatgaagtaaactaagctctaaaatcactgtgtcaatggcaagctgccaagaaccacagaagtggtcataaatatcttaaattaagggagctgaaagagagagtggaattaatcgaaaggctgcctcagcaagaaccataaagccataaattaactagcatcacacatccatgacgtcctccatgggatgagataaggtcatcgagcttcttccacgtggaaacctctttgtggtgctaacgatgaggctagcatcaaaagaattcccaccttcatgtggacagaatagtgtaatgtcatcacttatcgtatcgaacggtcaaattccgctcattatcaactccactgatttgtgaccgtaggatacgagggggacaagttgaagcactatatataggaagtgacagtaaagcgagttcagaatatcacatttatagggagcactgcatagcttacaagcttgtaagag

SEQ ID NO: 32 - DNA, 1800 bp, Populus trichocarpa
cattcaaataaataagagattttaggagggaatcatatcagcatccaacctatccatcaattttaatcggatcccagaactacaaattaattttcccaattgaattcctaaaggattggacatttctcaattgagtattccaacaactatatttattggcattttccgttatttcatgcaataaatagcatgaaactattttgaaaacaatttgacatttttctattaaaaagctacaatgatgtcaaatgaaacaagaatttcctaacacgtgagaattttttttataaatttaatcattagaagttcataacacattggagtcgaaaacaaaattcttacaaaatcttcaccatctaagttgtccatagcaaattacattttttacaagagcgatagcgtttgaaaacgcgggataactcgctttctaaaaagttttaatttttgttttttgctaaaaattaaatttttttttatgttttgaattgttttgatacgctgatcttaaaaataatttttttaaaataaaaaaaattattttaatgtatttcaacatgaaaaatactttaaaaagtaatcacaatattctctcaaacaggtccaaaataaaaaaaagaaaaaaaaaaagaagagggagttgctgggctgtttagtaatgtgtaacggaggtgagattataatgggcggaaatggttttagcccgacttctctttagaaatagccatgggcccatggcattcttctttgggctaatttaaattgctagaccattttctttttttttccctaagaaagtactggaccagaccattaagaaccctgtccactccttgtttacttggccattttcaatgcatactgagctggatttgatcgttagaggacctaaagcattgcttacttgtgaccacgtattgctttaaattttgaggttttgagggtgttgttttttaaattattattttttatttagagttgtattaaaataatatttttttatttattaagatttattcttgacatcaaaataataataaaaaatataaaaaaataattttaaataaaaaaaagtaaattttcaatgatctttatttggatcgcaattccaaaccaatactaaaaaccagattgttgagctttcatgaatgttttggttttactttccataatgaaattgctccaactaaaaaaaaaaaggtaaaatctagtgaatccagccaaacctaattggttaactctaaatttttttattttaaatatgaaacaatattatttttaaaaaatcttattgacccaaattgacattaactttttttttatatatatatatatatgaagtaaactaagctctaaaatcactgtgtcaatggcaagctgccaagaaccacagaagtggtcataaatatcttaaattaagggagctgaaagagagagtggaattaatcgaaaggctgcctcagcaagaaccataaagccataaattaactagcatcacacatccatgacgtcctccatgggatgagataaggtcatcgagcttcttccacgtggaaacctctttgtggtgctaacgatgaggctagcatcaaaagaattcccaccttcatgtggacagaatagtgtaatgtcatcacttatcgtatcgaacggtcaaattccgctcattatcaactccactgatttgtgaccgtaggatacgagggggacaagttgaagcactatatataggaagtgacagtaaagcgagttcagaatatcacatttatagggagcactgcatagcttacaagcttgtaagag

SEQ ID NO: 33 - DNA, 30 bp, Artificial Sequence
  Synthetic construct
gcagagctcggggaatgataggctgacaag

SEQ ID NO: 34 - DNA, 32 bp, Artificial Sequence
  Synthetic construct
gcaactagtcccaaaatcttgcctacttccat

SEQ ID NO: 35 - DNA, 53 bp, Artificial Sequence
  Synthetic construct
gggacaagtttgtacaaaaaagcaggcttaatggccgtaagtttcgtaacaac

SEQ ID NO: 36 - DNA, 53 bp, Artificial Sequence
  Synthetic construct
ggggaccactttgtacaagaaagctgggtcttagatgggtttggtgagccaat